(19) United States
(12) Patent Application Publication
(10) Pub. No.: US 2024/0392321 A1
(43) Pub. Date: Nov. 28, 2024

(54) BLIND EDITING OF POLYNUCLEOTIDE SEQUENCES

(71) Applicant: BIOCONSORTIA, INC., Davis, CA (US)

(72) Inventors: JOHN MALIN, SACRAMENTO, CA (US); DAMIAN CURTIS, DAVIS, CA (US); THOMAS WILLIAMS, WOODLAND, CA (US)

(21) Appl. No.: 18/689,000

(22) PCT Filed: Sep. 8, 2022
(86) PCT No.: PCT/US22/76096

§ 371 Date: Mar. 4, 2024

Related U.S. Application Data
(60) Provisional application No. 63/242,279, filed on Sep. 9, 2021.

(51) Int. Cl. C12N 15/90; C12N 15/70; C12N 15/74

(52) U.S. Cl. CPC C12N 15/902; C12N 15/70; C12N 15/74

(57) ABSTRACT

Compositions and methods are provided for the editing of a polynucleotide target sequence, for example, in the genome of a cell, such as a bacterial cell, without a priori knowledge of the target sequence to be edited. Also provided are libraries of target sequences, libraries of microbes comprising the same, and kits comprising the same.

### CROSS-REFERENCE TO RELATED APPLICATIONS

This application claims the benefit under 35 U.S.C. 119 (e) of U.S. Provisional Patent Application Ser. No. 63/242,279 filed 9 Sep. 2021, herein incorporated by reference in its entirety.

### FIELD

The disclosure relates to the field of molecular biology, in particular to compositions and methods for modifying polynucleotides, for example, in the genome of a cell.

### BACKGROUND

Recombinant DNA technology has made it possible to insert DNA sequences at targeted genomic locations and/or modify specific endogenous chromosomal sequences. Site-specific integration techniques, which employ site-specific recombination systems, as well as other types of recombination technologies, have been used to generate targeted modifications, such as insertions of genes of interest, in a variety of organisms. Genome-editing techniques such as designer zinc finger nucleases (ZFNs) or transcription activator-like effector nucleases (TALENs), or homing meganucleases, are available for producing targeted genome perturbations, but these systems tend to have a low specificity and employ designed nucleases that need to be redesigned for each target site, which renders them costly and time-consuming to prepare. More recent methods include guided RNA-endonucleases, such as CRISPR Cas endonucleases, for precise editing of a known target site; however, like the other known editing methods, it requires a priori knowledge of the target sequence, in order to effect site cleavage and modification.

Although several approaches have been developed to target a specific site for modification in the genome of an organism, there still remains a need for more effective genome engineering technologies that are affordable, easy to set up, scalable, and amenable to targeting sites for which the exact sequences may not be known, for editing similar sequences across different organisms, or targeting multiple positions within a genome.

### SUMMARY

Unlike other technologies, the genome modification methods described herein do not require a priori knowledge of the exact sequence of a target polynucleotide site.

Taking advantage of high levels of homology between target sequences, for example, in strains of the same species, or between sequences that naturally share high similarity (e.g., alleles, paralogs, orthologs, homologs), editing with a homology template can be incorporated into multiple recipients.

In some aspects, a method is provided for editing one or a plurality of target sites in a recipient polynucleotide. In some cases, the one or a plurality of target sites may be in the same organism or in different organisms.

In some aspects, a method of introducing an edit into a target site of a recipient polynucleotide is provided, the method comprising: determining the composition of a source polynucleotide; designing an introductory polynucleotide, wherein the introductory polynucleotide comprises a region that shares sufficient homology with at least 100, between 100 and 150, at least 150, between 150 and 200, at least 200, between 200 and 250, at least 250, between 250 and 300, at least 300, or greater than 300 nucleotides of the source polynucleotide of (a); wherein at least one of the following conditions is true: the sequence of recipient polynucleotide is undetermined, and/or the sequence of recipient polynucleotide is not 100% identical to the sequence of source polynucleotide; providing the introductory polynucleotide to the recipient polynucleotide; incubating the introductory polynucleotide and the recipient polynucleotide under conditions suitable for recombining; and assessing the recipient polynucleotide for at least one edit at the target site. In some embodiments, the method further comprises sequencing the recipient polynucleotide. In some embodiments, the source polynucleotide and recipient polynucleotide are homologs, orthologs, or paralogs. In some embodiments, the introductory polynucleotide further comprises a polynucleotide of interest, wherein the polynucleotide of interest is flanked by the sequences sharing sufficient homology with at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 450, at least 500, between 500 and 550, at least 550, between 550 and 600, at least 600, between 600 and 650, at least 650, between 650 and 700, at least 700, between 700 and 750, at least 750, between 750 and 800, at least 800, between 800 and 850, at least 850, at least 900, between 900 and 950, at least 950, between 1000, at least 1000, or greater than 1000 nucleotides up- and down-stream, respectively, of the source polynucleotide. In some aspects, the sufficient homology is provided by at least 80%, between 80% and 85%, at least 85%, between 85% and 90%, at least 90%, between 90% and 91%, at least 91%, between 91% and 92%, at least 92%, between 92% and 93%, at least 93%, at least 93%, between 93% and 94%, at least 94%, between 94% and 95%, at least 95%, between 95% and 96%, at least 96%, between 96% and 97%, at least 97%, at least 97%, between 97% and 98%, at least 98%, between 98% and 99%, at least 99%, between 99% and 100% or 100% sequence similarity between the source polynucleotide and the target polynucleotide; in other embodiments, sufficient homology is evidenced by the outcome of homologous recombination, irrespective of length or percent identity.

In some aspects, the method further comprises a polynucleotide of interest, wherein the polynucleotide of interest comprises a heterologous polynucleotide that is inserted into a target site of the recipient polynucleotide. In some embodiments, the polynucleotide of interest comprises a polynucleotide modification template.

In some aspects of the method, the edit is selected from the group consisting of: insertion of at least one nucleotide, deletion of at least one nucleotide, replacement of at least one nucleotide, molecular alteration of at least one nucleotide, and any combination of the preceding.

In some aspects of the method, the edit results in the increased expression of a gene, the decreased expression of a gene, the inactivation of a gene, the knockout of a gene, or the expression of a new gene or gene fusion (non-naturally occurring combination of two or more polynucleotides that form a transcribable unit).

In some aspects of the method, the recipient polynucleotide is in the genome of a cell.

In some aspects of the method, the cell is a bacterium. In some embodiments, the bacterium is of the genus Paenibacillus. In some aspects, the method further comprises incubating the cell under conditions that facilitate growth and reproduction.

In some aspects of the method, a plurality of recipient polynucleotides is provided. In some embodiments, at least two of the plurality of recipient polynucleotides are in the genomes of different cells. In some embodiments, the different cells comprise bacterial cells. In some embodiments, each bacterial cell is of a different species. In some embodiments, each bacterial cell is of a different strain of the same species. In some embodiments, the plurality of cells comprises a consortia. In some embodiments, the plurality of cells comprises a plurality of cells in which some cells are from the same genus, species, and/or strain.

In some aspects of the method, a plurality of recipient polynucleotides is provided, wherein the plurality of recipient polynucleotides is in vitro.

In some aspects of the method, comprising a plurality of source polynucleotides is provided.

In some aspects, a synthetic composition is provided, comprising: an introductory polynucleotide, comprising a region that shares sufficient homology with at least 100 nucleotides of a source polynucleotide; and a plurality of recipient polynucleotides; wherein the plurality of recipient polynucleotides are not all identical; wherein the source polynucleotide is identified or obtained from a source organism that is of the same species as the organism from which at least one of the recipient polynucleotide(s) is (are) identified or obtained; wherein at least one of the following conditions is true: the sequence of at least one recipient polynucleotide is undetermined, and/or the sequence of at least one recipient polynucleotide is not 100% identical to the sequence of source polynucleotide. In some embodiments, the plurality of recipient polynucleotides are comprised within a plurality of cells. In some embodiments, the source polynucleotide is comprised within a cell. In some embodiments, the synthetic composition comprises a plurality of source polynucleotides.

### BRIEF DESCRIPTION OF THE DRAWINGS

The disclosure can be more fully understood from the following detailed description and the accompanying drawings, which form a part of this application.

FIG. 1 depicts a schematic of one aspect of the “blind editing” method. A delivery vector, comprising an edit (checkered box, denoted with an asterisk (*)) to be introduced into one or more recipient(s) polynucleotides, is flanked by sequences (vertical dashed line and horizontal dashed line). Recipient polynucleotides can be comprised within organisms, such as bacteria (cartoons in the figure). Sequences sharing unknown or non-identical sequence homology with the flanking regions of the donor vector are shown in various weights of vertical and horizontal lines, respectively. Those recipients sharing sufficient homology with the flanking regions of the donor vector will have the edit denoted with an asterisk (*) inserted. Recipients lacking sufficient homology with the flanking regions will not have the edit incorporated. The resulting polynucleotides in the edited recipient comprise a hybrid sequence in which the regions of the homology sequences proximal to the delivered edit comprise sequences originating from the donor polynucleotide, and the regions of the homology sequence distal to the delivered edit comprise sequences originating from the recipient polynucleotide. The relative lengths of the sections of sequences originating from either donor or recipient polynucleotide may vary depending on the precise location of the homologous recombination event.

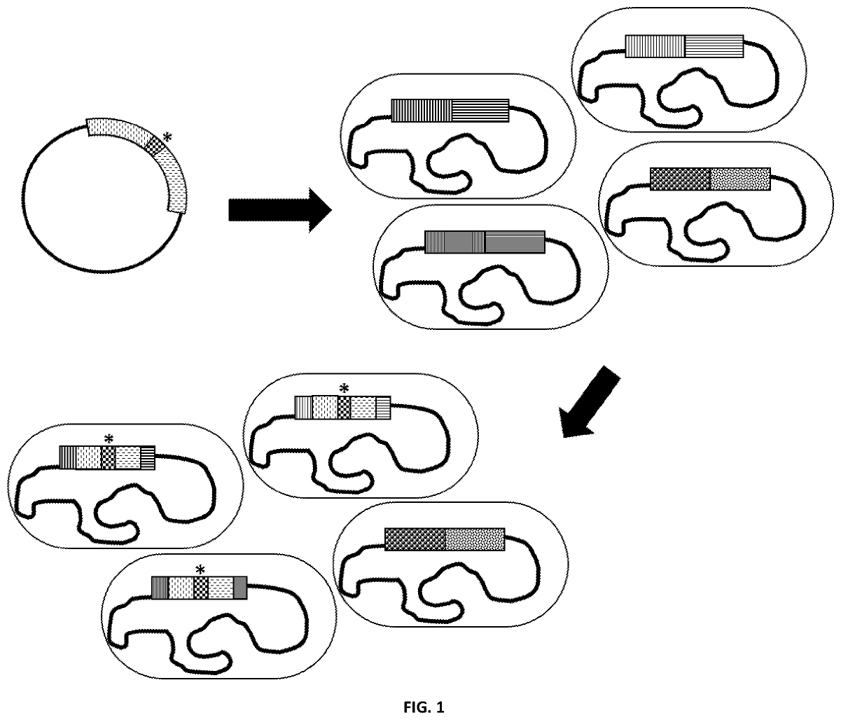

FIG. 2 depicts a schematic of one aspect of the “blind editing” method, in which a sequence is deleted from a recipient polynucleotide. Recipient polynucleotides can be comprised within organisms, such as bacteria (cartoons in the figure). Sequences sharing unknown or non-identical sequence homology with the flanking regions of the donor vector are shown in various weights of vertical and horizontal lines, respectively. Those recipients sharing sufficient homology with the flanking regions of the donor vector will have the sequences denoted with an asterisk (*) deleted. Recipients lacking sufficient homology with the flanking regions will not have the edit. The resulting polynucleotides of the edited recipient comprise a hybrid sequence in which the regions of the homology sequences proximal to the junction of the two homology sequences comprise sequences originating from the donor polynucleotide, and the regions of the homology sequence distal to the junction of the two homology sequences comprise sequences originating from the recipient polynucleotide. The relative lengths of the sections of sequences originating from either donor or recipient polynucleotide may vary depending on the precise location of the homologous recombination event.

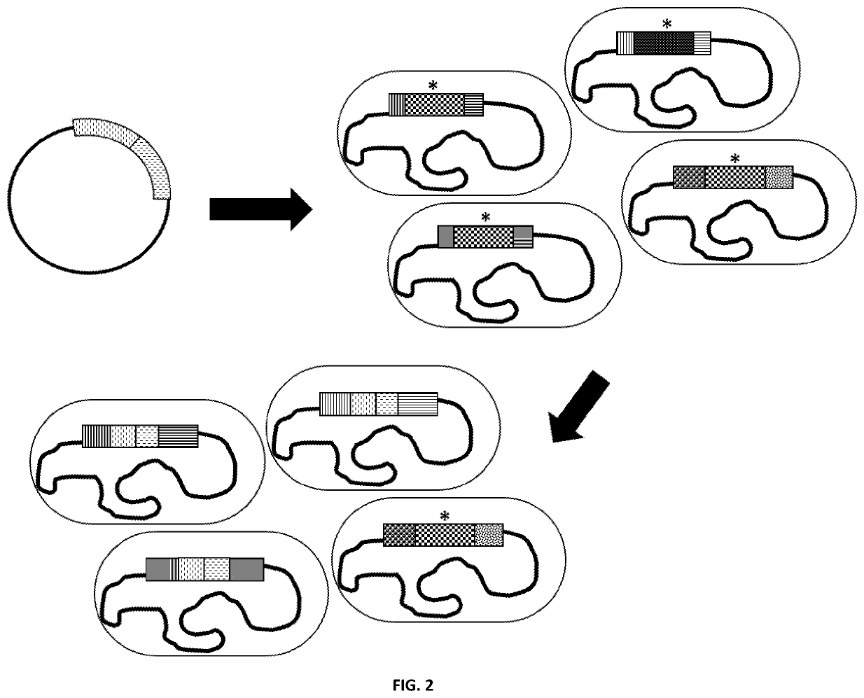

### DETAILED DESCRIPTION

Traditional methods for targeted genome editing require knowledge of a target site's polynucleotide sequence, and are often limited to a single edit of a single target. Multiplex editing of a plurality of target sites generally involves design of a plurality of individual molecules for the targeting and modification of the plurality of target sites.

Some types of homologous recombination in a cell require cellular mechanisms and/or enzymes to facilitate the repair of a break or insertion of a heterologous component. However, homologous recombination can occur strictly due to the homology present between a target and a donor polynucleotide introduced to the target.

Some target polynucleotides have the capability to tolerate some mismatch between the target and an introduced polynucleotide, resulting in homologous recombination in the absence of perfect homology. Disclosed herein are novel methods for accomplishing editing of a plurality of cells and/or polynucleotide target sites, leveraging the power of imperfect homologous recombination.

While the following terms are believed to be well understood by one of ordinary skill in the art, the following are set forth to facilitate explanation of the presently disclosed subject matter.

The term “a” or “an” refers to one or more of that entity, i.e., can refer to a plural referent. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

As used herein, the term “about” refers to up to ±10% of the recited value. For example, the term “about” can refer to ±1%, ±2%, ±3%, ±4%, ±5%, ±6%, ±7%, ±8%, ±9%, ±10%, of the recited value, or non-integer percentages thereof. By way of additional example, the term “about” may refer to ±0.2 minutes with respect to the retention times recited herein.

As used herein the terms “microorganism” or “microbe” should be taken broadly. These terms are used interchangeably and include, but are not limited to, the two prokaryotic domains, Bacteria and Archaea, as well as eukaryotic Fungi and Protists. As used herein, the term “microbe” or “microorganism” refers to any species or taxon of microorganism, including, but not limited to, Archaea, bacteria, microalgae, fungi (including mold and yeast species), mycoplasmas, microspores, nanobacteria, oomycetes, and protozoa. In some embodiments, a microbe or microorganism encompasses individual cells (e.g., unicellular microorganisms), or more than one cell (e.g., multi-cellular microorganism), or organisms that can be either multicellular or unicellular during the life cycle.

The term “microbial community” means a group of microbes comprising two or more genera, and/or species, and/or or strains. Unlike microbial consortia, a microbial community does not have to be carrying out a common function, or does not have to be participating in, or leading to, or correlating with, a recognizable parameter or plant phenotypic trait. The community may comprise one or more species, or strains of a species, of microbes. In some instances, the microbes coexist within the community symbiotically.

The term “microbial consortia” or “microbial consortium” refers to a subset of a microbial community of individual microbes, which can be described as carrying out a common function, or can be described as participating in, or leading to, or correlating with, a recognizable parameter or plant phenotypic trait. Consortia of microbes identified herein can each provide different aspects of a desired outcome (e.g., plant biotic stress control), and/or can work with one another in an additive fashion (e.g., one microbe providing control of one biotic stressor and another microbe providing control of a different biotic stressor), and/or can work with each other in a synergistic fashion (e.g., two or more microbes providing a level of biotic stress control to a plant greater than the sum of any individual microbe's effect).

A “population” of compositions, such as microbes, refers to a plurality of said compositions that are spatially and/or temporally co-located.

As used herein, the term “bacterium” or “bacteria” refers in general to any prokaryotic organism, and may reference an organism from either Kingdom Eubacteria (Bacteria), Kingdom Archaebacteria (Archaea), or both. In some cases, bacterial genera, species, or other taxonomic classifications have been reassigned due to various reasons (such as but not limited to the evolving field of whole genome sequencing), and it is understood that such nomenclature reassignments are within the scope of any claimed taxonomy. For example, certain species of the genus Erwinia have been described in the literature as belonging to genus Pantoea (Zhang, Y., Qiu, S. Examining phylogenetic relationships of Erwinia and Pantoea species using whole genome sequence data. Antonie van Leeuwenhoek 108, 1037-1046 (2015).).

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid”, “nucleotide sequence”, and “polynucleotide” are used interchangeably.

The term “genome” refers to the entire complement of genetic material (genes and non-coding sequences) that is present in each cell of an organism, or virus or organelle; and/or a complete set of chromosomes inherited as a unit from a parental organism. The term “genome” as it applies to a prokaryotic and eukaryotic cell or organism cells encompasses not only chromosomal DNA found within the nucleus, but organelle DNA found within subcellular components (e.g., mitochondria, or plastid) of the cell, as well as genetic material not incorporated into a chromosome or organelle, as well as stably-introduced plasmids.

“Extracellular” DNA and RNA encompasses polynucleotides that originated from a particular cell of an organism, such as a microbe.

As used herein, the term “genotype” refers to the genetic makeup of an individual cell, cell culture, tissue, organism (e.g., a plant), or group of organisms.

As used herein, the term “trait” refers to a characteristic or phenotype. For example, in the context of some embodiments of the present disclosure, yield of a crop relates to the amount of biomass produced by a plant (e.g., fruit, fiber, grain). Desirable traits may also include other plant characteristics, including but not limited to: water use efficiency, nutrient use efficiency, production, mechanical harvestability, fruit maturity, shelf life, pest/disease resistance, early plant maturity, tolerance to stresses, etc. A trait may be inherited in a dominant or recessive manner, or in a partial or incomplete-dominant manner. A trait may be monogenic (i.e., determined by a single locus) or polygenic (i.e., determined by more than one locus) or may also result from the interaction of one or more genes with the environment.

As used herein, the term “phenotype” refers to the observable characteristics of an individual cell, cell culture, organism (e.g., a plant), or group of organisms which results from the interaction between that individual's genetic makeup (i.e., genotype) and the environment.

As used herein, a “genomic region” is a portion of a polynucleotide, for example, a segment of a chromosome in the genome of a cell, that is present on either side of a target site or, alternatively, also comprises all or a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters. “Native gene” refers to a gene as found in its natural endogenous location with its own regulatory sequences.

An “allele” is one of several alternative forms of a gene occupying a given locus on a chromosome or genomic element. When all the alleles present at a given locus are the same, that organism is homozygous at that locus. If the alleles present at a given locus differ, that organism is heterozygous at that locus.

“Coding sequence” refers to a polynucleotide sequence which codes for a specific amino acid sequence.

“Non-coding sequences” include sequences such as “regulatory sequences”, which refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises a molecular alteration of one or more nucleotides. A mutated bacterium is a bacterium comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system.

The terms “knock-out”, “gene knock-out” and “genetic knock-out” are used interchangeably herein. A knock-out represents a DNA sequence of a cell that has been rendered partially or completely inoperative; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell (for example, by homologous recombination (HR), wherein a suitable donor DNA polynucleotide is also used). examples of knock-ins are a specific insertion of a heterologous amino acid coding sequence in a coding region of a gene, or a specific insertion of a transcriptional regulatory element in a genetic locus.

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which any composition of the disclosure may recognize, bind, cleave, nick, and/or integrate. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, “donor polynucleotide” is a construct that comprises a polynucleotide of interest to be inserted into the target site.

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

A “promoter” is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate or repress promoter activity, and may be an innate element of the promoter or a heterologous element inserted to enhance or repress the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example, by chemical compounds (chemical inducers) or in response to environmental, hormonal, chemical, and/or developmental signals.

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished using standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

By the term “endogenous” it is meant a sequence or other molecule that naturally occurs in a cell or organism. In one aspect, an endogenous polynucleotide is normally found in the genome of a cell; that is, not heterologous. In general, “heterologous” refers to a state conferred by the non-naturally-occurring association of one composition (e.g., chemical, molecule, seed, plant, gene) with another of the same or different type. A heterologous state may be defined as a composition (e.g., a gene) that is not in its naturally-occurring environment—e.g., different location within a genome, presence in a different organism. A composition that is in a heterologous combination with another composition is thus non-naturally-occurring, and may synonymously be referred to as a “synthetic combination”.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

As used herein, a “synthetic nucleotide sequence” or “synthetic polynucleotide sequence” is a nucleotide sequence that is not known to occur in nature or that is not naturally occurring Generally, such a synthetic nucleotide sequence will comprise at least one nucleotide difference when compared to any other naturally occurring nucleotide sequence.

As used herein, the term “homologous” or “homologue”, “homolog”, or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences.

These terms describe the relationship between a gene found in one species, subspecies, variety, cultivar or strain and the corresponding or equivalent gene in another species, subspecies, variety, cultivar or strain. For purposes of this disclosure homologous sequences are compared. “Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.), ALIGN Plus (Scientific and Educational Software, Pennsylvania) and AlignX (Vector NTI, Invitrogen, Carlsbad, CA). Another alignment program is Sequencher (Gene Codes, Ann Arbor, Michigan), using default parameters.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have the necessary structural similarity to act as substrates for homologous recombination. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having, for example, 90%, 95%, 98%, 99%, or 100% sequence identity, and percent sequence identity over a portion of the length of the sequences.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC-3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. It is understood by those of skill in the art that modifications to the preceding conditions may be made to test and/or validate hybridization, and still be within the scope of the disclosure.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=S and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6. 1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix-Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases. “BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

As used herein, the term “nucleotide change” refers to, e.g., nucleotide substitution, deletion, insertion, chemical alteration, or any of the preceding, as is well understood in the art.

As used herein, the term “protein modification” refers to, e.g., amino acid substitution, amino acid modification, deletion, and/or insertion, as is well understood in the art.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. In some cases, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

“Sequence identity” or “identity” in the context of nucleic acid or polypeptide sequences refers to the nucleic acid bases or amino acid residues in two sequences that are the same when aligned for maximum correspondence over a specified comparison window.

As used herein, the term “at least a portion” or “fragment” of a nucleic acid or polypeptide means a portion having the minimal size characteristics of such sequences, or any larger fragment of the full length molecule, up to and including the full length molecule. The term “fragment” refers to a contiguous set of nucleotides or amino acids. In one embodiment, a fragment is 2, 3, 4, 5, 6, 78, 9, 10, 11, 12, 13, 14, 15, 16, 17, 18, 19, 20, or greater than 20 contiguous nucleotides. In one embodiment, a fragment is 2, 3, 4, 5, 6, 78, 9, 10, 11, 12, 13, 14, 15, 16, 17, 18, 19, 20, or greater than 20 contiguous amino acids. A fragment may or may not exhibit the function of a sequence sharing some percent identity over the length of said fragment. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full length polypeptide. The length of the portion to be used will depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified organism.

The term “primer” as used herein refers to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

The terms “stringency” or “stringent hybridization conditions” refer to hybridization conditions that affect the stability of hybrids, e.g., temperature, salt concentration, pH, formamide concentration and the like. These conditions are empirically optimized to maximize specific binding and minimize non-specific binding of primer or probe to its target nucleic acid sequence. The terms as used include reference to conditions under which a probe or primer will hybridize to its target sequence, to a detectably greater degree than other sequences (e.g., at least 2-fold over background). Stringent conditions are sequence dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength and pH) at which 50% of a complementary target sequence hybridizes to a perfectly matched probe or primer. Typically, stringent conditions will be those in which the salt concentration is less than about 1.0 M Nation, typically about 0.01 to 1.0 M Na⁺ ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides).

Stringent conditions for hybridization may also be achieved with the addition of destabilizing agents such as formamide. Exemplary low stringent conditions or “conditions of reduced stringency” include hybridization with a buffer solution of 30% formamide, 1 M NaCl, 1% SDS at 37° C. and a wash in 2×SSC at 40° C. Exemplary high stringency conditions include hybridization in 50% formamide, 1M NaCl, 1% SDS at 37° C., and a wash in 0.1×SSC at 60° C. Hybridization procedures are well known in the art and are described by e.g., Ausubel et al., 1998 and Sambrook et al., 2001. In some embodiments, stringent conditions are hybridization in 0.25 M Na2HPO4 buffer (pH 7.2) containing 1 mM Na2EDTA, 0.5-20% sodium dodecyl sulfate at 45° C., such as 0.5%, 1%, 2%, 3%, 4%, 5%, 6%, 7%, 8%, 9%, 10%, 11%, 12%, 13%, 14%, 15%, 16%, 17%, 18%, 19% or 20%, followed by a wash in 5×SSC, containing 0.1% (w/v) sodium dodecyl sulfate, at 55° C. to 65° C.

The term “16S” refers to the DNA sequence of the 16S ribosomal RNA (rRNA) sequence of a bacterium. 16S rRNA gene sequencing is a well-established method for studying phylogeny and taxonomy of bacteria.

As used herein, the term “fungus” or “fungi” refers in general to any organism from Kingdom Fungi. Historical taxonomic classification of fungi has been according to morphological presentation. Beginning in the mid-1800's, it was recognized that some fungi have a pleomorphic life cycle, and that different nomenclature designations were being used for different forms of the same fungus. In 1981, the Sydney Congress of the International Mycological Association laid out rules for the naming of fungi according to their status as anamorph, teleomorph, or holomorph (Taylor, J. W. One Fungus=One Name: DNA and fungal nomenclature twenty years after PCR. IMA Fungus 2, 113-120 (2011).). With the development of genomic sequencing, it became evident that taxonomic classification based on molecular phylogenetics did not align with morphological-based nomenclature (Shenoy, B. D., Jeewon, R. and Hyde, K. D. (2007). Impact of DNA sequence-data on the taxonomy of anamorphic fungi. Fungal Diversity 26:1-54.). As a result, in 2011 the International Botanical Congress adopted a resolution approving the International Code of Nomenclature for Algae, Fungi, and Plants (Melbourne Code) (2012), with the stated outcome of designating “One Fungus=One Name” (Hawksworth, D. L. Managing and coping with names of pleomorphic fungi in a period of transition. IMA Fungus 3, 15-24 (2012)).

The term “Internal Transcribed Spacer” (“ITS”) refers to the spacer DNA (non-coding DNA) situated between the small-subunit ribosomal RNA (rRNA) and large-subunit (LSU) rRNA genes in the chromosome or the corresponding transcribed region in the polycistronic rRNA precursor transcript. ITS gene sequencing is a well-established method for studying phylogeny and taxonomy of fungi. In some cases, the “Large SubUnit” (“LSU”) sequence is used to identify fungi. LSU gene sequencing is a well-established method for studying phylogeny and taxonomy of fungi. Some fungal microbes of the present invention may be described by an ITS sequence and some may be described by an LSU sequence. Both are understood to be equally descriptive and accurate for determining taxonomy.

As used herein, “isolate,” “isolated,” “isolated microbe,” and like terms, are intended to mean that the one or more microorganisms has been separated from at least one of the materials with which it is associated in a particular environment (for example, soil, water, plant tissue).

Thus, an “isolated microbe” does not exist in its naturally occurring environment; rather, it is through the various techniques described herein that the microbe has been removed from its natural setting and placed into a non-naturally occurring state of existence. Thus, the isolated strain may exist as, for example, a biologically pure culture, or as spores (or other forms of the strain) in association with an agricultural carrier.

In certain aspects of the disclosure, the isolated microbes exist as isolated and biologically pure cultures. It will be appreciated by one of skill in the art, that an isolated and biologically pure culture of a particular microbe, denotes that said culture is substantially free (within scientific reason) of other living organisms and contains only the individual microbe in question. The culture can contain varying concentrations of said microbe. The present disclosure notes that isolated and biologically pure microbes often “necessarily differ from less pure or impure materials.” See, e.g., In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H. K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

As used herein, “individual isolates” should be taken to mean a composition, or culture, comprising a predominance of a single genera, species, or strain, of microorganism, following separation from one or more other microorganisms. The phrase should not be taken to indicate the extent to which the microorganism has been isolated or purified. However, “individual isolates” can comprise substantially only one genus, species, or strain, of microorganism.

The term “growth medium” as used herein, is any medium which is suitable to support growth of a plant. By way of example, the media may be natural or artificial including, but not limited to: soil, potting mixes, bark, vermiculite, hydroponic solutions alone and applied to solid plant support systems, and tissue culture gels. It should be appreciated that the media may be used alone or in combination with one or more other media. It may also be used with or without the addition of exogenous nutrients and physical support systems for roots and foliage.

In one embodiment, the growth medium is a naturally occurring medium such as soil, sand, mud, clay, humus, regolith, rock, or water. In another embodiment, the growth medium is artificial. Such an artificial growth medium may be constructed to mimic the conditions of a naturally occurring medium; however, this is not necessary. Artificial growth media can be made from one or more of any number and combination of materials including sand, minerals, glass, rock, water, metals, salts, nutrients, water. In one embodiment, the growth medium is sterile. In another embodiment, the growth medium is not sterile.

The medium may be amended or enriched with additional compounds or components, for example, a component which may assist in the interaction and/or selection of specific groups of microorganisms with the plant and each other. For example, antibiotics (such as penicillin) or sterilants (for example, quaternary ammonium salts and oxidizing agents) could be present and/or the physical conditions (such as salinity, plant nutrients (for example, organic and inorganic minerals (such as phosphorus, nitrogenous salts, ammonia, potassium and micronutrients such as cobalt and magnesium), pH, and/or temperature) could be amended.

The term “plant” generically includes whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. A “plant element” is intended to reference either a whole plant or a plant component, which may comprise differentiated and/or undifferentiated tissues, for example, but not limited to plant tissues, parts, and cell types. In one embodiment, a plant element is one of the following: whole plant, seedling, meristematic tissue, ground tissue, vascular tissue, dermal tissue, seed, leaf, root, shoot, stem, flower, fruit, stolon, bulb, tuber, corm, keiki, shoot, bud, tumor tissue, and various forms of cells and culture (e.g., single cells, protoplasts, embryos, callus tissue). The term “plant organ” refers to plant tissue or a group of tissues that constitute a morphologically and functionally distinct part of a plant. As used herein, a “plant part” is synonymous to a “portion” of a plant, and refers to any part of the plant, and can include distinct tissues and/or organs, and may be used interchangeably with the term “tissue” throughout.

“Progeny” comprises any subsequent generation of an organism, produced via sexual or asexual reproduction.

As used herein, the term “plant element” refers to plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like, as well as the parts themselves. Grain is intended to mean the mature seed produced by commercial growers for purposes other than growing or reproducing the species. Progeny, variants, and mutants of the regenerated plants are also included within the scope of the invention, provided that these plants comprise the introduced polynucleotides.

Similarly, a “plant reproductive element” is intended to generically reference any part of a plant that is able to initiate other plants via either sexual or asexual reproduction of that plant, for example, but not limited to: seed, seedling, root, shoot, cutting, scion, graft, stolon, bulb, tuber, corm, keiki, or bud. The plant element may be in plant or in a plant organ, tissue culture, or cell culture.

The term “monocotyledonous” or “monocot” refers to the subclass of angiosperm plants also known as “Monocotyledoneae”, whose seeds typically comprise only one embryonic leaf, or cotyledon. The term includes references to whole plants, plant elements, plant organs (e.g., leaves, stems, roots, etc.), seeds, plant cells, and progeny of the same.

The term “dicotyledonous” or “dicot” refers to the subclass of angiosperm plants also knows as “Dicotyledoneae”, whose seeds typically comprise two embryonic leaves, or cotyledons. The term includes references to whole plants, plant elements, plant organs (e.g., leaves, stems, roots, etc.), seeds, plant cells, and progeny of the same.

As used herein, the term “cultivar” refers to a variety, strain, or race, of plant that has been produced by horticultural or agronomic techniques and is not normally found in wild populations.

As used herein, the term “molecular marker”, “marker”, or “genetic marker” refers to an indicator that is used in methods for assessing (e.g., visualizing) differences in characteristics of nucleic acid sequences. Examples of such indicators are restriction fragment length polymorphism (RFLP) markers, amplified fragment length polymorphism (AFLP) markers, single nucleotide polymorphisms (SNPs), insertion mutations, microsatellite markers (SSRs), sequence-characterized amplified regions (SCARs), cleaved amplified polymorphic sequence (CAPS) markers or isozyme markers or combinations of the markers described herein which defines a specific genetic and chromosomal location. Mapping of molecular markers in the vicinity of an allele is a procedure which can be performed by the average person skilled in molecular-biological techniques. A marker can also include gene expression that confers resistance or tolerance to an external substance, such as an antibiotic or herbicide. Visual markers, such as the incorporation of a fluorescent protein gene, is also contemplated.

As used herein, “improved” should be taken broadly to encompass improvement of a characteristic of a plant, as compared to a control plant, or as compared to a known average quantity associated with the characteristic in question. For example, “improved” plant biomass associated with application of a beneficial microbe, or consortia, of the disclosure can be demonstrated by comparing the biomass of a plant treated by the microbes taught herein to the biomass of a control plant not treated. Alternatively, one could compare the biomass of a plant treated by the microbes taught herein to the average biomass normally attained by the given plant, as represented in scientific or agricultural publications known to those of skill in the art. In the present disclosure, “improved” does not necessarily demand that the data be statistically significant (e.g., p<0.05); rather, any quantifiable difference demonstrating that one value (e.g., the average treatment value) is different from another (e.g., the average control value) can rise to the level of “improved.”

As used herein, “inhibiting and suppressing” and like terms should not be construed to require complete inhibition or suppression, although this may be desired in some embodiments.

The compositions and methods herein may provide for an improved trait of interest in a particular organism, for example but not limited to: in an animal cell, in a human cell, in a plant cell, in a bacterial cell.

The compositions and methods herein may provide for an improved “agronomic trait” or “trait of agronomic importance” or “trait of agronomic interest” to a plant, which may include, but not be limited to, the following: disease resistance, drought tolerance, heat tolerance, cold tolerance, salinity tolerance, metal tolerance, herbicide tolerance, improved water use efficiency, improved nitrogen utilization, improved associated nitrogen fixation, pest resistance, herbivore resistance, pathogen resistance, yield improvement, health enhancement, vigor improvement, growth improvement, photosynthetic capability improvement, nutrition enhancement, altered protein content, altered oil content, increased biomass, increased shoot length, increased root length, improved root architecture, modulation of a metabolite, modulation of the proteome, increased seed weight, altered seed carbohydrate composition, altered seed oil composition, altered seed protein composition, altered seed nutrient composition, as compared to an isoline plant not comprising a modification derived from the methods or compositions herein

“Agronomic trait potential” is intended to mean a capability of a plant element for exhibiting a phenotype, preferably an improved agronomic trait, at some point during its life cycle, or conveying said phenotype to another plant element with which it is associated in the same plant.

In some embodiments, the cell or organism has at least one heterologous trait. As used herein, the term “heterologous trait” refers to a phenotype imparted to a cell or organism by an exogenous molecule or other organism (e.g., a microbe), DNA segment, heterologous polynucleotide or heterologous nucleic acid.

Various changes in phenotype are of interest to the present disclosure, including but not limited to modifying the fatty acid composition in a plant, altering the amino acid content of a plant, altering a plant's pathogen defense mechanism, increasing a plant's yield of an economically important trait (e.g., grain yield, forage yield, etc.) and the like. These results can be achieved by providing expression of heterologous products or increased expression of endogenous products in plants using the methods and compositions of the present disclosure

A “synthetic combination” can include a combination of a plant and a microbe, or a plant and a composition, of the disclosure. The combination may be achieved, for example, by coating the surface of a seed of a plant, such as an agricultural plant, or host plant tissue (root, stem, leaf, etc.), with a microbe of the disclosure. Further, a “synthetic combination” can include a combination of microbes of various strains or species. Synthetic combinations have at least one variable that distinguishes the combination from any combination that occurs in nature. That variable may be, inter alia, a concentration of microbe on a seed or plant tissue that does not occur naturally, or a combination of microbe and plant that does not naturally occur, or a combination of microbes or strains that do not occur naturally together. In each of these instances, the synthetic combination demonstrates the hand of man and possesses structural and/or functional attributes that are not present when the individual elements of the combination are considered in isolation.

In some embodiments, a microbe can be “endogenous” to a seed or plant. As used herein, a microbe is considered “endogenous” to a plant or seed, if the microbe is derived from the plant specimen from which it is sourced. That is, if the microbe is naturally found associated with said plant. In embodiments in which an endogenous microbe is applied to a plant, then the endogenous microbe is applied in an amount that differs from the levels found on the plant in nature, differs in the persistence in the plant, differs in the consistency of association, or other aspect. Thus, in one example, a microbe that is endogenous to a given plant can still form a synthetic combination with the plant, if the microbe is present on said plant at a level that does not occur naturally.

In some embodiments, a composition (such as a microbe) can be “heterologous” (also termed “exogenous”) to another composition (such as a seed or plant), and in some aspects is referred to herein as a “heterologous composition”. As used herein, a microbe is considered “heterologous” to a plant or seed, if the microbe is not derived from the plant specimen from which it is sourced. That is, if the microbe is not naturally found associated with said plant. For example, a microbe that is normally associated with leaf tissue of a maize plant is considered exogenous to a leaf tissue of another maize plant that naturally lacks said microbe. In another example, a microbe that is normally associated with a maize plant is considered exogenous to a wheat plant that naturally lacks said microbe. A composition is “heterologously disposed” when mechanically or manually applied, artificially inoculated, associated with, or disposed onto or into a plant element, seedling, plant or onto or into a plant growth medium or onto or into a treatment formulation so that the treatment exists on or in the plant element, seedling, plant, plant growth medium, or formulation in a manner not found in nature prior to the application of the treatment, e.g., said combination which is not found in nature in that plant variety, at that stage in plant development, in that plant tissue, in that abundance, or in that growth environment (for example, drought). In some embodiments, such a manner is contemplated to be selected from the group consisting of: the presence of the microbe; presence of the microbe in a different number of cells, concentration, or amount; the presence of the microbe in a different plant element, tissue, cell type, or other physical location in or on the plant; the presence of the microbe at different time period, e.g., developmental phase of the plant or plant element, time of day, time of season, and combinations thereof. In some embodiments, “heterologously disposed” means that the microbe being applied to a different tissue or cell type of the plant element than that in which the microbe is naturally found. In some embodiments, “heterologously disposed” means that the microbe is applied to a developmental stage of the plant element, seedling, or plant in which said microbe is not naturally associated, but may be associated at other stages. For example, if a microbe is normally found at the flowering stage of a plant and no other stage, a microbe applied at the seedling stage may be considered to be heterologously disposed. In some embodiments, a microbe is heterologously disposed the microbe is normally found in the root tissue of a plant element but not in the leaf tissue, and the microbe is applied to the leaf. In another non-limiting example, if a microbe is naturally found in the mesophyll layer of leaf tissue but is being applied to the epithelial layer, the microbe would be considered to be heterologously disposed. In some embodiments, “heterologously disposed” means that the native plant element, seedling, or plant does not contain detectable levels of the microbe in that same plant element, seedling, or plant. In some embodiments, “heterologously disposed” means that the microbe being applied is at a greater concentration, number, or amount of the plant element, seedling, or plant, than that which is naturally found in said plant element, seedling, or plant. For example, a microbe is heterologously disposed when present at a concentration that is at least 1.5 times greater, between 1.5 and 2 times greater, 2 times greater, between 2 and 3 times greater, 3 times greater, between 3 and 5 times greater, 5 times greater, between 5 and 7 times greater, 7 times greater, between 7 and 10 times greater, 10 times greater, or even greater than 10 times higher number, amount, or concentration than the concentration that was present prior to the disposition of said microbe. In another non-limiting example, a microbe that is naturally found in a tissue of a cupressaceous tree would be considered heterologous to tissue of a maize, wheat, cotton, soybean plant. In another example, a microbe that is naturally found in leaf tissue of a maize, spring wheat, cotton, soybean plant is considered heterologous to a leaf tissue of another maize, spring wheat, cotton, soybean plant that naturally lacks said microbe, or comprises the microbe in a different quantity.

Microbes can also be “heterologously disposed” on a given plant tissue. This means that the microbe is placed upon a plant tissue that it is not naturally found upon. For instance, if a given microbe only naturally occurs on the roots of a given plant, then that microbe could be exogenously applied to the above-ground tissue of a plant and would thereby be “heterologously disposed” upon said plant tissue. As such, a microbe is deemed heterologously disposed, when applied on a plant that does not naturally have the microbe present or does not naturally have the microbe present in the number that is being applied.

The compositions and methods herein may provide for a “modulated” “agronomic trait” or “trait of agronomic importance” to a host plant, which may include, but not be limited to, the following: altered oil content, altered protein content, altered seed carbohydrate composition, altered seed oil composition, and altered seed protein composition, chemical tolerance, cold tolerance, delayed senescence, disease resistance, drought tolerance, ear weight, growth improvement, health enhancement, heat tolerance, herbicide tolerance, herbivore resistance, improved nitrogen fixation, improved nitrogen utilization, improved root architecture, improved water use efficiency, increased biomass, increased root length, increased seed weight, increased shoot length, increased yield, increased yield under water-limited conditions, kernel mass, kernel moisture content, metal tolerance, number of ears, number of kernels per ear, number of pods, nutrition enhancement, pathogen resistance, pest resistance, photosynthetic capability improvement, salinity tolerance, stay-green, vigor improvement, increased dry weight of mature seeds, increased fresh weight of mature seeds, increased number of mature seeds per plant, increased chlorophyll content, increased number of pods per plant, increased length of pods per plant, reduced number of wilted leaves per plant, reduced number of severely wilted leaves per plant, and increased number of non-wilted leaves per plant, a detectable modulation in the level of a metabolite, a detectable modulation in the level of a transcript, and a detectable modulation in the proteome, compared to an isoline plant grown from a seed without said seed treatment formulation. By the term “modulated”, it is intended to refer to a change in a characteristic, such as an agronomic trait, that is changed by virtue of the presence of the microbe(s), exudate, broth, metabolite, etc. In aspects, the modulation provides for the imparting of a trait, such as a trait of agronomic or other importance.

“Introducing” or “providing” is intended to mean presenting to a target, such as a cell or organism, a polynucleotide or polypeptide or polynucleotide-protein complex, in such a manner that the component(s) gains access to the interior of a cell of the organism or to the cell itself. In some aspects, “introducing” or “providing” means presenting one composition to another, such that they are in physical proximity.

A “polynucleotide of interest” includes any nucleotide sequence encoding a protein or polypeptide that improves desirability of crops, i.e. a trait of agronomic interest. Polynucleotides of interest include, but are not limited to: polynucleotides encoding important traits for agronomics, herbicide-resistance, insecticidal resistance, disease resistance, nematode resistance, herbicide resistance, microbial resistance, fungal resistance, viral resistance, fertility or sterility, grain characteristics, commercial products, phenotypic marker, or any other trait of agronomic or commercial importance. A polynucleotide of interest may additionally be utilized in either the sense or anti-sense orientation. Further, more than one polynucleotide of interest may be utilized together, or “stacked”, to provide additional benefit.

The terms “decreased,” “fewer,” “slower” and “increased” “faster” “enhanced” “greater” as used herein refers to a decrease or increase in a characteristic of the modified plant element or resulting plant compared to an unmodified plant element or resulting plant. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more lower than the untreated control and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the untreated control.

As used herein, the term “before”, in reference to a sequence position, refers to an occurrence of one sequence upstream, or 5′, to another sequence.

The meaning of abbreviations is as follows: “sec” means second(s), “min” means minute(s), “h” means hour(s), “d” means day(s), “μL” means microliter(s), “mL” means milliliter(s), “L” means liter(s), “μM” means micromolar, “mM” means millimolar, “M” means molar, “mmol” means millimole(s), “μmole” or “umole” mean micromole(s), “g” means gram(s), “μg” or “ug” means microgram(s), “ng” means nanogram(s), “U” means unit(s), “bp” means base pair(s) and “kb” means kilobase(s).

### Microbes and Microorganisms

As used herein the term “microorganism” should be taken broadly. It includes, but is not limited to, prokaryotic Bacteria and Archaea, as well as eukaryotic Fungi and Protists.

By way of example, the microorganisms may include: Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium, Variovorax and Halomonas), Firmicutes (such as Bacillus, Paenibacillus, Lactobacillus, Mycoplasma, and Acetobacterium), Actinobacteria (such as Brevibacterium, Janibacter, Streptomyces, Rhodococcus, Microbacterium, Curtobacterium, Cellulomonas, and Nocardioides), and the fungi Ascomycota (such as Trichoderma, Ampelomyces, Coniothyrium, Paecoelomyces, Penicillium, Cladosporium, Hypocrea, Beauveria, Metarhizium, Verticillium, Cordyceps, Pichea, and Candida), Basidiomycota (such as Coprinus, Corticium, and Agaricus) and Oomycota (such as Pythium), and Mucoromycota (such as Mucor, and Mortierella); as well as Orbilia/Arthrobotrys, Lysinibacillus, Microbacterium, Talaromyces, Arthrobacter, Kosakonia, Masillia, Novosphingobium, and Tumebacillus, as well as Arbuscular mycorrhizal Fungi such as Glomeromycota.

In a particular embodiment, the microorganism is an endophyte, and/or an epiphyte, and/or a microorganism inhabiting the plant rhizosphere, rhizoplane, or rhizosheath, and/or phyllosphere, and/or phylloplane. That is, the microorganism may be found present in the soil material adhered to or within the roots, shoots, and/or leaves of a plant or in the area immediately adjacent a plant's above ground or below ground surfaces, or any combination of the above.

In one embodiment, the microorganism is an endophyte. Endophytes may benefit host plants by preventing pathogenic organisms from colonizing them, for producing certain compounds such as IAA or volatile organic compounds (VOCs), for providing nitrogen fixation to enhance plant health, or other benefits. Extensive colonization of the plant tissue by endophytes creates a “barrier effect,” where the local endophytes outcompete and prevent pathogenic organisms from taking hold. Endophytes may also produce chemicals which inhibit the growth of competitors, including pathogenic organisms.

In certain embodiments, the microorganism is unculturable. This should be taken to mean that the microorganism is not known to be culturable or is difficult to culture using methods known to one skilled in the art.

Microorganisms of the present disclosure may be collected or obtained from any source or contained within and/or associated with material collected from any source.

In an embodiment, the microorganisms are obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish)); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, road surfaces).

In another embodiment the microorganisms are collected from a source likely to favor the selection of appropriate microorganisms. By way of example, the source may be a particular environment in which it is desirable for other plants to grow, or which is thought to be associated with terroir. In another example, the source may be a plant having one or more desirable traits, for example, a plant which naturally grows in a particular environment or under certain conditions of interest. In other cases, microbes may be selected from samples with neutral or undesirable traits, or combinations of traits. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the microorganisms may be collected from commercial crops grown in such environments, or more specifically from individual crop plants best displaying a trait of interest amongst a crop grown in any specific environment, for example, the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste, or smell. The microorganisms may be collected from a plant of interest or any material occurring in the environment of interest, including fungi and other animal and plant biota, soil, water, sediments, and other elements of the environment as referred to previously. In certain embodiments, the microorganisms are individual isolates separated from different environments.

In one embodiment, a microorganism or a combination of microorganisms, of use in the methods of the disclosure may be selected from a pre-existing collection of individual microbial species or strains based on some knowledge of their likely or predicted benefit to a plant. For example, the microorganism may be predicted to: improve nitrogen fixation; release phosphate and/or potassium and/or silica and/or zinc and/or iron from the soil organic matter; release phosphate and/or potassium and/or silica and/or zinc and/or iron from an inorganic form (e.g., rock); “fix carbon” in the root microsphere; live in the rhizosphere of the plant thereby assisting the plant in absorbing nutrients from the surrounding soil and then providing these more readily to the plant; increase the number of nodules on the plant roots and thereby increase the number of symbiotic nitrogen fixing bacteria (e.g., Rhizobium species) per plant and the amount of nitrogen fixed for the plant and/or macroogransisms and/or microorganisms; elicit plant defensive responses such as ISR (induced systemic resistance) or SAR (systemic acquired resistance) which help the plant resist the invasion and spread of pathogenic microorganisms; compete with microorganisms deleterious to plant growth or health by antagonism, or competitive utilization of resources such as nutrients or space; change the color of one or more part of the plant, or change the chemical profile of the plant, its smell, taste, or one or more other quality. In other cases, the microorganism can assist the plant in growing in extreme or unfavorable conditions, such as high salinity, high heavy metal content, extreme temperature, etc.

In one embodiment a microorganism or combination of microorganisms is selected from a pre-existing collection of individual microbial species or strains that provides no knowledge of their likely or predicted benefit to a plant. For example, a collection of unidentified microorganisms isolated from plant tissues without any knowledge of their ability to improve plant growth or health, or a collection of microorganisms collected to explore their potential for producing compounds that could lead to the development of pharmaceutical drugs.

In one embodiment, the microorganisms are acquired from the source material (for example, soil, rock, water, air, dust, plant or other organism) with or within which they naturally reside. The microorganisms may be provided in any appropriate form, having regard to its intended use in the methods of the disclosure. However, by way of example only, the microorganisms may be provided as an aqueous suspension, gel, homogenate, granule, powder, slurry, live organism, or dried material.

The microorganisms of the disclosure may be isolated in substantially pure or mixed cultures. They may be concentrated, diluted, or provided in the natural concentrations in which they are found in the source material. For example, microorganisms from saline sediments may be isolated for use in this disclosure by suspending the sediment in fresh water and allowing the sediment to fall to the bottom. The water containing the bulk of the microorganisms may be removed by decantation after a suitable period of settling and either applied directly to the plant growth medium, or concentrated by filtering or centrifugation, diluted to an appropriate concentration and applied to the plant growth medium with the bulk of the salt removed. By way of further example, microorganisms from mineralized or toxic sources may be similarly treated to recover the microbes for application to the plant growth material to minimize the potential for damage to the plant.

In another embodiment, the microorganisms are used in a crude form, in which they are not isolated from the source material in which they naturally reside. For example, the microorganisms are provided in combination with the source material in which they reside; for example, as soil, or the roots, seed, or foliage of a plant. In this embodiment, the source material may include one or more species of microorganisms.

In some embodiments, a mixed population of microorganisms is used in the methods of the disclosure.

In embodiments of the disclosure where the microorganisms are isolated from a source material (for example, the material in which they naturally reside), any one or a combination of a number of standard techniques which will be readily known to skilled persons may be used. However, by way of example, these in general employ processes by which a solid or liquid culture of a single microorganism can be obtained in a substantially pure form, usually by physical separation on the surface of a solid microbial growth medium or by volumetric dilutive isolation into a liquid microbial growth medium. These processes may include isolation from dry material, liquid suspension, slurries or homogenates in which the material is spread in a thin layer over an appropriate solid gel growth medium, or serial dilutions of the material made into a sterile medium and inoculated into liquid or solid culture media.

Whilst not essential, in one embodiment, the material containing the microorganisms may be pre-treated prior to the isolation process in order to either multiply all microorganisms in the material, or select portions of the microbial population, either by enriching the material with microbial nutrients (for example, by heating the sample to select for microorganisms resistant to heat exposure (for example, bacilli), or by exposing the sample to low concentrations of an organic solvent or sterilant (for example, household bleach) to enhance the survival of spore-forming or solvent-resistant microorganisms). Microorganisms can then be isolated from the enriched materials or materials treated for selective survival, as above.

In an embodiment of the disclosure, endophytic or epiphytic microorganisms are isolated from plant material. Any number of standard techniques known in the art may be used and the microorganisms may be isolated from any appropriate tissue in the plant, including for example, root, stem and leaves, and plant reproductive tissues. By way of example, conventional methods for isolation from plants typically include the sterile excision of the plant material of interest (e.g., root or stem lengths, leaves), surface sterilization with an appropriate solution (e.g., 2% sodium hypochlorite), after which the plant material is placed on nutrient medium for microbial growth (See, for example, Strobel G and Daisy B (2003) Microbiology and Molecular Biology Reviews 67 (4): 491-502; Zinniel D K et al. (2002) Applied and Environmental Microbiology 68 (5): 2198-2208).

In one embodiment of the disclosure, the microorganisms are isolated from plant tissue. Further methodology for isolating microorganisms from plant material are detailed hereinafter.

In one embodiment, the microbial population is exposed (prior to the method or at any stage of the method) to a selective pressure. For example, exposure of the microorganisms to heating before their addition to a plant growth medium (preferably sterile) is likely to enhance the probability that the plants selected for a desired trait will be associated with spore-forming microbes that can more easily survive in adverse conditions, in commercial storage, or if applied to seed as a coating, in an adverse environment.

In certain embodiments, as mentioned herein before, the microorganism(s) may be used in crude form and need not be isolated from a plant or a media. For example, plant material or growth media which includes the microorganisms identified to be of benefit to a selected plant may be obtained and used as a crude source of microorganisms for the next round of the method or as a crude source of microorganisms at the conclusion of the method. For example, whole plant material could be obtained and optionally processed, such as mulched or crushed.

Alternatively, individual tissues or parts of selected plants (such as leaves, stems, roots, and seeds) may be separated from the plant and optionally processed, such as mulched or crushed. In certain embodiments, one or more part of a plant which is associated with the second set of one or more microorganisms may be removed from one or more selected plants and, where any successive repeat of the method is to be conducted, grafted on to one or more plant used in any step of the plant breeding methods.

### Sourcing of Microbes

The microbes of the present disclosure were obtained, among other places, at various locales in New Zealand and the United States

### Isolation and Culturing of Microbes

In some cases, microbes were identified by utilizing standard microscopic techniques to characterize the microbes' phenotype, which was then utilized to identify the microbe to a taxonomically recognized species. In some cases, microbes were identified using sequencing and phenotyping techniques standard in the art.

The isolation, identification, and culturing of the microbes of the present disclosure can be effected using standard microbiological techniques. Examples of such techniques may be found in Gerhardt, P. (ed.) Methods for General and Molecular Microbiology. American Society for Microbiology, Washington, D.C. (1994) and Lennette, E. H. (ed.) Manual of Clinical Microbiology, Third Edition. American Society for Microbiology, Washington, D.C. (1980), each of which is incorporated by reference.

Isolation can be effected by streaking the specimen on a solid medium (e.g., nutrient agar plates) to obtain a single colony, which is characterized by the phenotypic traits described hereinabove (e.g., Gram positive/negative, capable of forming spores aerobically/anaerobically, cellular morphology, carbon source metabolism, acid/base production, enzyme secretion, metabolic secretions, etc.) and to reduce the likelihood of working with a culture which has become contaminated.

For example, for isolated bacteria of the disclosure, biologically pure isolates can be obtained through repeated subculture of biological samples, each subculture followed by streaking onto solid media to obtain individual colonies. Methods of preparing, thawing, and growing lyophilized bacteria are commonly known, for example, Gherna, R. L. and C. A. Reddy. 2007. Culture Preservation, p 1019-1033. In C. A. Reddy, T. J. Beveridge, J. A. Breznak, G. A. Marzluf, T. M. Schmidt, and L. R. Snyder, eds. American Society for Microbiology, Washington, D.C., 1033 pages; herein incorporated by reference. Thus freeze-dried liquid formulations and cultures stored long term at −70° C. in solutions containing glycerol are contemplated for use in providing formulations of the present inventions.

The bacteria of the disclosure can be propagated in a “culture medium”, which may comprise a liquid medium or solid medium, under aerobic conditions. Medium for growing the bacterial strains of the present disclosure includes a carbon source, a nitrogen source, and inorganic salts, as well as specially required substances such as vitamins, amino acids, nucleic acids and the like. Examples of suitable carbon sources which can be used for growing the bacterial strains include, but are not limited to, starch, peptone, yeast extract, amino acids, sugars such as glucose, arabinose, mannose, glucosamine, maltose, and the like; salts of organic acids such as acetic acid, fumaric acid, adipic acid, propionic acid, citric acid, gluconic acid, malic acid, pyruvic acid, malonic acid and the like; alcohols such as ethanol and glycerol and the like; oil or fat such as soybean oil, rice bran oil, olive oil, corn oil, sesame oil. The amount of the carbon source added varies according to the kind of carbon source and is typically between 1 to 100 gram(s) per liter of medium. Preferably, glucose, starch, and/or peptone is contained in the medium as a major carbon source, at a concentration of 0.1-5% (W/V). Examples of suitable nitrogen sources which can be used for growing the bacterial strains of the present invention include, but are not limited to, amino acids, yeast extract, tryptone, beef extract, peptone, potassium nitrate, ammonium nitrate, ammonium chloride, ammonium sulfate, ammonium phosphate, ammonia or combinations thereof. The amount of nitrogen source varies according to the type of nitrogen source, typically between 0.1 to 30 gram per liter of medium. The inorganic salts, potassium dihydrogen phosphate, dipotassium hydrogen phosphate, disodium hydrogen phosphate, magnesium sulfate, magnesium chloride, ferric sulfate, ferrous sulfate, ferric chloride, ferrous chloride, manganous sulfate, manganous chloride, zinc sulfate, zinc chloride, cupric sulfate, calcium chloride, sodium chloride, calcium carbonate, sodium carbonate can be used alone or in combination. The amount of inorganic acid varies according to the kind of the inorganic salt, typically between 0.001 to 10 gram per liter of medium. Examples of specially required substances include, but are not limited to, vitamins, nucleic acids, yeast extract, peptone, meat extract, malt extract, dried yeast and combinations thereof. Cultivation can be effected at a temperature, which allows the growth of the bacterial strains, essentially, between 20° C. and 46° C. In some aspects, a temperature range is 30° C.-37° C. For optimal growth, in some embodiments, the medium can be adjusted to pH 7.0-7.4. It will be appreciated that commercially available media may also be used to culture the bacterial strains, such as Nutrient Broth or Nutrient Agar available from Difco, Detroit, MI. It will be appreciated that cultivation time may differ depending on the type of culture medium used and the concentration of sugar as a major carbon source.

In some aspects, cultivation lasts between 24-96 hours. Bacterial cells thus obtained are isolated from culture media using methods that are well known in the art. Examples include, but are not limited to, membrane filtration and centrifugal separation. The pH may be adjusted using and the culture may be dried using a freeze dryer, until the water content becomes equal to about 4% or less. Microbes may also be prepared as a suspension concentrate.

Microbial co-cultures may be obtained by propagating each strain as described hereinabove. It will be appreciated that the microbial strains may be cultured together when compatible culture conditions can be employed. In other cases, microbial strains may be combined after incubation.

### Identification of Microbes

Microbes can be distinguished into a Genus based on polyphasic taxonomy, which incorporates all available phenotypic and genotypic data into a consensus classification (Vandamme et al. 1996. Polyphasic taxonomy, a consensus approach to bacterial systematics. Microbiol Rev 1996, 60:407-438). One accepted genotypic method for defining species is based on overall genomic relatedness, such that strains which share approximately 70% or more relatedness using DNA-DNA hybridization, with 5° C. or less ΔTm (the difference in the melting temperature between homologous and heterologous hybrids), under standard conditions, are considered to be members of the same species. Another method includes genomic Average Nucleotide Identity (ANI) with cut-off values typically 96% but some variants have lower cut-offs (Ciufo S, Kannan S, Sharma S, et al. Using average nucleotide identity to improve taxonomic assignments in prokaryotic genomes at the NCBI. Int J Syst Evol Microbiol. 2018; 68 (7): 2386-2392).

Thus, populations that share greater than the aforementioned 70% threshold can be considered to be variants of the same species.

For bacterial microbes, the 16S rRNA sequences are often used for determining taxonomy and making distinctions between species, in that if a 16S rRNA sequence shares less than a specified % sequence identity from a reference sequence, then the two organisms from which the sequences were obtained are said to be of different species.

Thus, one could consider microbes to be of the same species, if they share at least 80%, 85%, 90%, 95%, 97%, 98%, or 99% sequence identity across the 16S or 16S rRNA or rDNA sequence. In some aspects, a microbe could be considered to be the same species only if it shares at least 95% identity.

Further, one could define microbial strains of a species, as those that share at least 80%, 85%, 90%, 95%, 97%, 98%, or 99% sequence identity across the 16S rRNA sequence.

Comparisons may also be made with 23S rRNA sequences against reference sequences. In some aspects, a microbe could be considered to be the same strain only if it shares at least 95% identity. In some embodiments, “substantially similar genetic characteristics” means a microbe sharing at least 95% identity.

For fungal microbes, the ITS (Internal Transcriber Sequence) is often used for identification of taxonomy. Among the regions of the ribosomal cistron, the internal transcribed spacer (ITS) region has the highest probability of successful identification for the broadest range of fungi, with the most clearly defined barcode gap between inter- and intraspecific variation, and has been proposed as the formal fungal identification sequence (Schoch et al., PNAS Apr. 17, 2012 109 (16) 6241-6246). Other loci, such as 18S or 28S are also used for fungal identification.

Unculturable microbes often cannot be assigned to a definite species in the absence of a phenotype determination, the microbes can be given a candidatus designation within a genus provided their 16S rRNA sequences subscribes to the principles of identity with known species.

One approach is to observe the distribution of a large number of strains of closely related species in sequence space and to identify clusters of strains that are well resolved from other clusters. This approach has been developed by using the concatenated sequences of multiple core (house-keeping) genes to assess clustering patterns, and has been called multilocus sequence analysis (MLSA) or multilocus sequence phylogenetic analysis. MLSA has been used successfully to explore clustering patterns among large numbers of strains assigned to very closely related species by current taxonomic methods, to look at the relationships between small numbers of strains within a genus, or within a broader taxonomic grouping, and to address specific taxonomic questions. More generally, the method can be used to ask whether bacterial species exist—that is, to observe whether large populations of similar strains invariably fall into well-resolved clusters, or whether in some cases there is a genetic continuum in which clear separation into clusters is not observed.

In order to more accurately make a determination of genera, a determination of phenotypic traits, such as morphological, biochemical, and physiological characteristics are made for comparison with a reference genus archetype. The colony morphology can include color, shape, pigmentation, production of slime, etc. Features of the cell are described as to shape, size, Gram reaction, extracellular material, presence of endospores, flagella presence and location, motility, and inclusion bodies. Biochemical and physiological features describe growth of the organism at different ranges of temperature, pH, salinity and atmospheric conditions, growth in presence of different sole carbon and nitrogen sources. One of ordinary skill in the art would be reasonably apprised as to the phenotypic traits that define the genera of the present disclosure. For instance, colony color, form, and texture on a particular agar (e.g., YMA) can be used to identify species of Rhizobium.

In one embodiment, bacterial microbes taught herein were identified utilizing 16S rRNA gene sequences. It is known in the art that 16S rRNA contains hypervariable regions that can provide species/strain-specific signature sequences useful for bacterial identification. In the present disclosure, many of the microbes were identified via partial (500-1200 bp) 16S rRNA sequence signatures. In aspects, each strain represents a pure colony isolate that was selected from an agar plate. Selections were made to represent the diversity of organisms present based on any defining morphological characteristics of colonies on agar medium. The medium used, in embodiments, was R2A, PDA, Nitrogen-free semi-solid medium, or MRS agar. Colony descriptions of each of the ‘picked’ isolates were made after 24-hour growth and then entered into our database. Sequence data was subsequently obtained for each of the isolates.

Phylogenetic analysis using the 16S rRNA gene was used to define “substantially similar” species belonging to common genera and also to define “substantially similar” strains of a given taxonomic species. Further, we recorded physiological and/or biochemical properties of the isolates that can be utilized to highlight both minor and significant differences between strains that could lead to advantageous behavior on plants.

### Microbial Consortia

In aspects, the disclosure provides microbial consortia comprising a combination of at least any two microbes or isolates.

In certain embodiments, the consortia of the present disclosure comprise two microbes, or three microbes, or four microbes, or five microbes, or six microbes, or seven microbes, or eight microbes, or nine microbes, or ten or more microbes. Said microbes of the consortia are different microbial species, or different strains of a microbial species, or different isolates of the same strain, or different mutants of the same strain, or different edited variants of the same strain.

In some embodiments, the disclosure provides consortia, in which some or all of the microbes comprise an edited genome.

### Polynucleotide Target Site Modification

A polynucleotide target site may be comprised within a genome or may be isolated from its naturally-occurring state.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989). Transformation methods are well known to those skilled in the art and are described infra. Transformation methods are well known to those skilled in the art.

Vectors and constructs include circular plasmids, and linear polynucleotides, comprising a polynucleotide of interest and optionally other components including linkers, adapters, regulatory or analysis. In some examples a recognition site and/or target site can be comprised within an intron, coding sequence, 5′ UTRs, 3′ UTRs, and/or regulatory regions.

Various methods and compositions can be employed to obtain a cell or organism having a polynucleotide of interest inserted in a target site. A polynucleotide of interest may be inserted into another polynucleotide, for example, into the genome of an organism, by various methods.

A heterologous (in some aspects, synthetic) polynucleotide may be introduced to a target site. Such an “introductory polynucleotide” comprises, at a minimum, regions sharing at least 25% to 100% sequence identity, for example, at least 80% identity, between 80% and 85%, at least 85%, between 85% and 90%, at least 90%, between 90% and 91%, at least 91%, between 91% and 92%, at least 92%, between 92% and 93%, at least 93%, between 93% and 94%, at least 94%, between 94% and 95%, at least 95%, between 95% and 96%, at least 96%, between 96% and 97%, at least 97%, between 97% and 98%, at least 98%, between 98% and 99%, at least 99%, between 99% and 100%, or 100% identity with a sequence at or near the target site. The region can be a length of at least 50, between 50 and 100, at least 100, between 100 and 150, at least 150, between 150 and 200, at least 200, between 200 and 250, at least 250, between 250 and 300, at least 300, between 300 and 350, between 350 and 400, at least 400, between 400 and 450, at least 450, between 450 and 500, at least 500, between 500 and 550, at least 550, between 550 and 600, at least 600, between 650 and 700, at least 700, between 700 and 750, at least 750, between 750 and 800, at least 800, between 800 and 850, at least 850, between 950 and 1000, at least 1000, or even greater than 1000 contiguous nucleotides in length.

Cells include, but are not limited to, eukaryotic, prokaryotic, bacterial, protist, fungal, yeast, non-conventional yeast, animal, mammalian, human, plant, insect, and plant cells.

### Double-Strand Break-Mediated Polynucleotide Modification

In general, DNA targeting can be performed by cleaving one or both strands at a specific polynucleotide sequence. Once a single or double-strand break is induced in the DNA, the cell's DNA repair mechanism is activated to repair the break via nonhomologous end-joining (NHEJ) or Homology-Directed Repair (HDR) processes which can lead to modifications at the target site.

Once a single or double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements (such as chromosomal translocations) are possible (Siebert and Puchta, 2002, Plant Cell 14:1121-31; Pacher et al., 2007, Genetics 175:21-9).

DNA double-strand breaks appear to be an effective factor to stimulate homologous recombination pathways (Puchta et al., (1995) Plant Mol Biol 28:281-92; Tzfira and White, (2005) Trends Biotechnol 23:567-9; Puchta, (2005) J Exp Bot 56:1-14). Using DNA-breaking agents, a two- to nine-fold increase of homologous recombination was observed between artificially constructed homologous DNA repeats in plants (Puchta et al., (1995) Plant Mol Biol 28:281-92).

Alternatively, Homology-Directed Repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called Homologous Recombination (HR), which has the longest sequence homology requirements between the donor and acceptor DNA. Other forms of HDR include single-stranded annealing (SSA) and breakage-induced replication, which require shorter sequence homology relative to HR. Homology-directed repair at nicks (single-stranded breaks) can occur via a mechanism distinct from HDR at double-strand breaks (Davis and Maizels. 2014 PNAS (0027-8424), 111 (10), p. E924-E932).

A repair of a polynucleotide double-strand break by the cell's NHEJ and/or HDR pathways can result in the perfect re-annealing of the target strand, resulting in no change in the composition. Alternatively, one or more modifications may be introduced by the repair mechanism itself with no additional molecule, by purposeful integration of a heterologous component (e.g., a donor polynucleotide), or by a template-directed repair (e.g., a polynucleotide modification template). The resulting modification can be a mutation, nucleotide substitution, nucleotide chemical alteration, nucleotide deletion, nucleotide insertion, or any combination or plurality of the preceding. The impact of any of those modification(s) to a cell comprising the modified polynucleotide may be, for example, but not limited to, a knockout of a gene, an increase or decrease in the expression of a gene, or the expression of a new gene.

Assays to measure the single or double-strand break of a target site are known in the art and generally measure the overall activity and specificity of the agent on DNA substrates comprising recognition sites.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments (multiplexing).

### Homologous Recombination

Homologous Recombination, or HR, is the exchange of genetic material between two strands of DNA that contain regions of similar base sequences. Homologous recombination occurs naturally in eukaryotic organisms, bacteria, and certain viruses and is a powerful tool in genetic engineering.

HR-directed polynucleotide modification may be accomplished via enzymes such as recombinases, or may occur as a function of sequence similarity between the target and the introduced (donor) polynucleotide.

Alteration of the genome of a prokaryotic or eukaryotic cell or organism cell through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28: e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al., 1992, Recombinant DNA, 2nd Ed., Scientific American Books distributed by WH Freeman & Co.).

Various methods and compositions can be employed to obtain a cell or organism having a polynucleotide of interest inserted in a target site. Such methods can employ homologous recombination (HR) to provide integration of the polynucleotide of interest at the target site. In one method described herein, a polynucleotide of interest is introduced into the organism cell via a donor polynucleotide construct.

The donor polynucleotide construct further comprises a first and a second region of homology that flank the polynucleotide of interest. The first and second regions of homology of the donor polynucleotide share homology to a first and a second genomic region, respectively, present in or flanking the target site of the cell or organism genome.

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also be described by percent sequence identity over the full aligned length of the two polynucleotides which includes percent sequence identity at least of about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, between 98% and 99%, 99%, between 99% and 100%, or 100%. Sufficient homology includes any combination of polynucleotide length, global percent sequence identity, and optionally conserved regions of contiguous nucleotides or local percent sequence identity that can result in homologous recombination, for example, sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology-Hybridization with Nucleic Acid Probes, (Elsevier, New York).

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some instances the regions of homology share significant sequence homology to the genomic sequence immediately flanking the target site, it is recognized that the regions of homology can be designed to have sufficient homology to regions that may be further 5′ or 3′ to the target site. The regions of homology can also have homology with a fragment of the target site along with downstream genomic regions

In one embodiment, the first region of homology further comprises a first fragment of the target site and the second region of homology comprises a second fragment of the target site, wherein the first and second fragments are dissimilar.

### Polynucleotide Editing

The methods and compositions included herein are useful for editing the composition of polynucleotide sequences that may effect phenotypic changes in an organism comprising such.

Polynucleotide edits include, but are not limited to, modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates comprising target sites.

The nucleotide to be edited can be located within or outside a target site, and may be present in vitro, or as part of the genomic complement of a host cell. Modification of a target sequence may be in the form of a nucleotide insertion, a nucleotide deletion, a nucleotide substitution, the addition of an atom molecule to an existing nucleotide, a nucleotide modification, or the binding of a heterologous polynucleotide or polypeptide to said target sequence. Any or all of the possible polynucleotide components may be provided as part of a vector, a construct, a linearized or circularized plasmid, or as part of a chimeric molecule. Each component may be provided to the reaction mixture separately or together. In some aspects, one or more of the polynucleotide components are operably linked to a heterologous noncoding regulatory element that regulates its expression. Incubation conditions will vary according to desired outcome. The temperature is preferably at least 10 degrees Celsius, between 10 and 15, at least 15, between 15 and 17, at least 17, between 17 and 20, at least 20, between 20 and 22, at least 22, between 22 and 25, at least 25, between 25 and 27, at least 27, between 27 and 30, at least 30, between 30 and 32, at least 32, between 32 and 35, at least 35, at least 36, at least 37, at least 38, at least 39, at least 40, or even greater than 40 degrees Celsius. The time of incubation is at least 1 minute, at least 2 minutes, at least 3 minutes, at least 4 minutes, at least 5 minutes, at least 6 minutes, at least 7 minutes, at least 8 minutes, at least 9 minutes, at least 10 minutes, or even greater than 10 minutes.

The storage buffer of any one of the components, or the reaction mixture, may be optimized for stability, efficacy, or other parameters. Additional components of the storage buffer or the reaction mixture may include a buffer composition, Tris, EDTA, dithiothreitol (DTT), phosphate-buffered saline (PBS), sodium chloride, magnesium chloride, HEPES, glycerol, BSA, a salt, an emulsifier, a detergent, a chelating agent, a redox reagent, an antibody, nuclease-free water, a proteinase, and/or a viscosity agent. In some aspects, the storage buffer or reaction mixture further comprises a buffer solution with at least one of the following components: HEPES, MgCl2, NaCl, EDTA, a proteinase, Proteinase K, glycerol, nuclease-free water.

### Uses in Microbiology, Agriculture, Pharmaceuticals, and Medical Research

The presently disclosed methods may be used to modify polynucleotides, including polynucleotides in vivo, ex vivo, in vitro. In vivo modification may include polynucleotides comprised within a cell. Cells include, but are not limited to, human, non-human, animal, mammalian, bacterial, fungal, insect, yeast, non-conventional yeast, and plant cells as well as plants and seeds produced by the methods described herein.

Bacterial cells may include either Gram-positive or Gram-negative bacteria, for example but not limited to: Paenibacillus, Klebsiella, Kosakonia, Bacillus.

Any plant can be used with the compositions and methods described herein, including monocot and dicot plants, and plant elements.

Animal cells can include, but are not limited to: an organism of a phylum including chordates, arthropods, mollusks, annelids, cnidarians, or echinoderms; or an organism of a class including mammals, insects, birds, amphibians, reptiles, or fishes. In some aspects, the animal is human, mouse, C. elegans, rat, fruit fly (Drosophila spp.), zebrafish, chicken, dog, cat, guinea pig, hamster, chicken, Japanese ricefish, sea lamprey, pufferfish, tree frog (e.g., Xenopus spp.), monkey, or chimpanzee. Particular cell types that are contemplated include haploid cells, diploid cells, reproductive cells, neurons, muscle cells, endocrine or exocrine cells, epithelial cells, muscle cells, tumor cells, embryonic cells, hematopoietic cells, bone cells, germ cells, somatic cells, stem cells, pluripotent stem cells, induced pluripotent stem cells, progenitor cells, meiotic cells, and mitotic cells. In some aspects, a plurality of cells from an organism may be used.

Genome modification may be used to effect a genotypic and/or phenotypic change on the target organism. Such a change is preferably related to an improved phenotype of interest or a physiologically-important characteristic, the correction of an endogenous defect, or the expression of some type of expression marker. In some aspects, the phenotype of interest or physiologically-important characteristic is related to the overall health, fitness, or fertility of the organism, the ecological fitness of the organism, or the relationship or interaction of the organism with other organisms or abiotic factors in its environment.

Any one or more of the compositions disclosed herein, useful for in vitro or in vivo polynucleotide detection, binding, and/or modification, may be comprised within a kit.

Certain aspects of the present disclosure, include, without limitation:
Aspect 1: A method of introducing an edit into a target site of a recipient polynucleotide, the method comprising: determining the composition of a source polynucleotide; designing an introductory polynucleotide, wherein the introductory polynucleotide comprises a region that shares sufficient homology with at least 100 nucleotides of the source polynucleotide of (a); wherein at least one of the following conditions is true: the sequence of recipient polynucleotide is undetermined, and/or the sequence of recipient polynucleotide is not 100% identical to the sequence of source polynucleotide; providing the introductory polynucleotide to the recipient polynucleotide; incubating the introductory polynucleotide and the recipient polynucleotide under conditions suitable for recombining; and assessing the recipient polynucleotide for at least one edit at the target site.
Aspect 2: The method of Aspect 1, further comprising: sequencing the recipient polynucleotide.
Aspect 3: The method of Aspect 1, wherein the source polynucleotide and recipient polynucleotide are alleles, homologs, orthologs, or paralogs.
Aspect 4: The method of Aspect 1, wherein the source polynucleotide and recipient polynucleotide are comprised within the same organism.
Aspect 5: The method of Aspect 1, wherein the composition of the source polynucleotide is determined by generating a consensus sequence from a plurality of sequences.
Aspect 6: The method of Aspect 1, wherein the introductory polynucleotide further comprises a polynucleotide of interest, wherein the polynucleotide of interest is flanked by the sequences sharing sufficient homology with at least 100 nucleotides up- and down-stream, respectively, of the source polynucleotide.
Aspect 7: The method of Aspect 6, wherein the polynucleotide of interest comprises a heterologous polynucleotide that is inserted into a target site of the recipient polynucleotide.
Aspect 8: The method of Aspect 6, wherein the polynucleotide of interest comprises a polynucleotide modification template.
Aspect 9: The method of Aspect 1, wherein the edit is selected from the group consisting of: insertion of at least one nucleotide, deletion of at least one nucleotide, replacement of at least one nucleotide, molecular alteration of at least one nucleotide, and any combination of the preceding.
Aspect 10: The method of Aspect 1, wherein the edit results in the increased expression of a gene, the decreased expression of a gene, the inactivation of a gene, the knockout of a gene, the expression of a new gene, or the fusion of genes.
Aspect 11: The method of Aspect 1, wherein sufficient homology is at least 80% identity over at least 100 nucleotides.
Aspect 12: The method of Aspect 1, wherein sufficient homology is determined by successful homologous recombination between the recipient polynucleotide and the introduced polynucleotide.
Aspect 13: The method of Aspect 1, wherein the recipient polynucleotide is in the genome of a cell.
Aspect 14: The method of Aspect 15, wherein the cell is a bacterium.
Aspect 15: The method of Aspect 16, wherein the bacterium is of the genus Paenibacillus.
Aspect 16: The method of Aspect 16, wherein the bacterium is of the species Paenibacillus polymyxa.
Aspect 17: The method of Aspect 15, further comprising: incubating the cell under conditions that facilitate growth and reproduction.
Aspect 18: The method of Aspect 1, comprising a plurality of recipient polynucleotides.
Aspect 19: The method of Aspect 18, wherein at least two of the plurality of recipient polynucleotides are in the genomes of different cells.
Aspect 20: The method of Aspect 19, wherein the different cells comprise bacterial cells.
Aspect 21: The method of Aspect 20, wherein each bacterial cell is of a different species.
Aspect 22: The method of Aspect 20, wherein each bacterial cell is of a different strain of the same species.
Aspect 23: The method of Aspect 18, wherein the plurality of recipient polynucleotides are in vitro.
Aspect 24: The method of Aspect 1, comprising a plurality of source polynucleotides.
Aspect 25: A synthetic composition, comprising: an introductory polynucleotide, comprising a region that shares sufficient homology with at least 100 nucleotides of a source polynucleotide; and a plurality of recipient polynucleotides;
Aspect 26: wherein the plurality of recipient polynucleotides are not all identical;
Aspect 27: wherein the source polynucleotide is identified or obtained from a source organism that is of the same species as the organism from which at least one of the recipient polynucleotide(s) is (are) identified or obtained;
Aspect 28: wherein at least one of the following conditions is true: the sequence of at least one recipient polynucleotide is undetermined, and/or the sequence of at least one recipient polynucleotide is not 100% identical to the sequence of source polynucleotide.
Aspect 29: The synthetic composition of Aspect 25, wherein the plurality of recipient polynucleotides are comprised within a plurality of cells.
Aspect 30: The synthetic composition of Aspect 25, wherein the source polynucleotide is comprised within a cell.
Aspect 31: The synthetic composition of Aspect 25, comprising a plurality of source polynucleotides.
Aspect 32: A synthetic composition, comprising: a plurality of introductory polynucleotides, each comprising a region that shares sufficient homology with at least 100 nucleotides of a source polynucleotide; and one or more recipient polynucleotide(s);
Aspect 33: wherein the plurality of source polynucleotides are not all identical;
Aspect 34: wherein the source polynucleotide is: identified or obtained from a source organism that is of the same species as the organism from which at least one of the recipient polynucleotide(s) is (are) identified or obtained; or obtained from a consensus sequence of a plurality of polynucleotides;
Aspect 35: wherein at least one of the following conditions is true: the sequence of at least one recipient polynucleotide is undetermined, and/or the sequence of at least one recipient polynucleotide is not 100% identical to the sequence of at least one source polynucleotide.
Aspect 36: A kit comprising: a plurality of introductory polynucleotides, each comprising a region that shares sufficient homology with at least 100 nucleotides of a source polynucleotide, wherein the plurality of introductory polynucleotides are not all identical; and a composition that promotes stability of the introductory polynucleotides.
Aspect 37: The kit of Aspect 24, wherein the source polynucleotide is identified, obtained, or derived from a Paenibacillus species or strain.

While the invention has been particularly shown and described with reference to a preferred embodiment and various alternate embodiments, it will be understood by persons skilled in the relevant art that various changes in form and details can be made therein without departing from the spirit and scope of the invention. Various alterations, modifications, and improvements of the present disclosure that readily occur to those skilled in the art, including certain alterations, modifications, substitutions, and improvements are also part of this disclosure. For instance, while the particular examples below may illustrate the methods and embodiments described herein using a specific plant, the principles in these examples may be applied to any plant. Therefore, it will be appreciated that the scope of this invention is encompassed by the embodiments recited herein rather than solely by the specific examples that are exemplified below.

All cited patents and publications referred to in this application are herein incorporated by reference in their entirety, for all purposes, to the same extent as if each were individually and specifically incorporated by reference.

### EXAMPLES

The following examples demonstrate the successful implementation of homologous recombination for the “blind” editing of target sites, for which the exact sequence may not have been determined for all. Although the specific implementation of these methods was performed with target sites from the genome of bacteria, these methods may be successfully used to accomplish single and multiplex homologous recombination-produced edits in any polynucleotide sequence, isolated or within an organism, that shares sufficient homology with regions of the sequence from which the source polynucleotide is identified.

Briefly, a source bacterial strain was isolated and a genomic sequence obtained, using methods known in the art. A target site was selected for editing, for example, insertion of a heterologous polynucleotide. Primers were designed and synthesized for that target site.

Different strains of the same species, as well as different strains of different species, were identified for use as recipient strains.

All recipient strains were transformed with the same plasmid, comprising an antibiotic selectable marker, and a donor polynucleotide (comprising a polynucleotide of interest flanked by sequences sharing 100% identity with approximately 1000 nucleotides near the target site of genomic sequence of the source strain, called the “homology region(s)”).

Strains were incubated under conditions suitable for recombining, and successful transconjugants were plated onto antibiotics at high temperature (to prevent the plasmid from replicating). Only those strains that had successful integration of the donor polynucleotide grew. Final confirmation of successful editing was confirmed by PCR, using primers designed for the source strain but outside the sequence selected for the homology region(s).

These methods are disclosed as exemplary embodiments, but one of skill in the art would understand that equivalent approaches (e.g., other incubation conditions, other temperatures, other selectable markers or schema) would be considered as alternative embodiments and within the scope of the instant disclosure.

### Example 1: Cloning

The sequences approximately 1000 bp upstream and downstream (“homology regions”) of the last six nucleic acids of a selected genomic site were amplified from genomic DNA prepared from an overnight liquid culture of a source strain via polymerase chain reaction (PCR) using Q5 Hot Start polymerase. Amplification primers were designed to append Gibson assembly overhangs allowing for assembly into a shuttle vector digested with the restriction enzymes BamHI and EcoRI and the addition of the nucleic acid sequence “ATCGAT” between the upstream and downstream homology fragments. The purified upstream and downstream PCR products were combined with the shuttle vector digested with BamHI and EcoRI and Gibson assembly reagent and incubated at 50° C. for 60 minutes. The assembly was transformed into chemically competent Echericha coli DH5a via heat shock and recovered on LB agar +100 μg/mL ampicillin plates. Colonies were selected for overnight growth in LB+100 ug/mL ampicillin and the plasmids were extracted, and the proper assembly was confirmed via restriction digestion. Plasmids that yielded the appropriate banding patterns were sequenced to confirm that no unwanted changes to the genomic sequence were introduced during cloning. The plasmid was then transformed into 2,3-diaminopropionic acid (DAP) auxotroph donor strain E. coli BW29427 electrocompetent cells via electroporation, yielding the donor strain, comprising the introductory polynucleotide (that which was for introduction into the recipient strain(s)). Only the plasmid(s) with the desire edit was constructed and used. The introductory polynucleotide comprised homology regions.

Although the homology regions selected for these experiments comprised 1000 nucleotides up- and down-stream of the genomic site sequence, it is contemplated that shorter regions of homology may also be utilized successfully, for example, at least 250 nucleotides. Shorter homology regions may reduce the number of successful conjugants, but high-throughput screening methods allow for rapid, efficient, and cost-effective identification.

### Example 2: Transformation by Conjugation

The donor strain was inoculated into LB+100 μg/mL ampicillin, 300 mM DAP and grown overnight at 37° C. Recipient strains (Paenibacillus spp.) were inoculated into TSB from fresh R2A agar plates and grown overnight at 30° C. 1 mL of the overnight culture of the donor strain was aliquoted into 2 mL microcentrifuge tubes. The cells were pelleted by centrifugation at 3000×g for 5 minutes. The supernatant was discarded, and the cells were resuspended in 1 mL of the recipient strain overnight culture. The cell mixture was pelleted by centrifugation at 3000×g for 5 minutes, the supernatant was discarded, and the cell mixture was resuspended in 100 ul of sterile water. The resuspended cell mixture was spotted onto LB+300 mM DAP plates. The plates were incubated overnight at 25° C., then the cell mixture was gently resuspended from the surface of the agar in 1 mL sterile water using a sterile 1-spreader. The resuspended cells were collected in 2 mL microcentrifuge tubes and pelleted by centrifugation at 3000×g for 5 minutes. The supernatant was removed and discarded, and the cells were resuspended in 100 ul of sterile water. The cells were spread over the surface of a TSA+1 ug/mL Erythromycin, 25 ug/mL Lincomycin (MLS) plate. The plates were incubated at 25° C., and transconjugants were recovered from the plates 24-48 hours later.

### Example 3: Integration and Excision

Transconjugants were inoculated into TSB+MLS and grown overnight at 25° C. Dilutions of the liquid cultures were plated onto TSA+MLS plates and incubated overnight at 37° C. Recovered colonies at the high temperature indicated putative integration of the plasmid. Integration was confirmed for some putative integrants via PCR using primers sets that align to the junction of the native genomic DNA outside of the homology region and the plasmid backbone. Integrated colonies were inoculated into TSB+MLS and grown overnight at 37° C. 5 ul of the overnight culture was subcultured into 5 mL TSB without antibiotics and grown overnight at 25° C. to induce the excision of the plasmid backbone. 5 ul of the cultures were further subcultured into 5 mL fresh TSB and grown over night at 25° C. two more times, for three total rounds of excision subculturing without antibiotics. Dilutions of the final excision subcultures were plated onto R2A plates without antibiotics and grown overnight at 25° C. Recovered colonies were replica plated onto TSB+MLS to assay for loss of antibiotic resistance, indicating excision and loss of the plasmid backbone.

Colonies that were susceptible to MLS were selected for confirmation of successful editing. Loss of antibiotic resistance indicated either successful plasmid excision or reversion to wild type. To assay for proper editing, the edit regions were amplified via PCR and sequenced by Sanger sequencing.

Results from cloning, transformation, integration, and excision of various types of donor plasmids (comprising a variety of different types of edits) in 44 different strains of Paenibacillus are shown in Table 1.

TABLE 1
Results

| | | | | | | | | PCR | | | |
|---|---|---|---|---|---|---|---|---|---|---|---|
| | Recipient | Editing | | Source | | | | confirmation | Loss of | | |
| Recipient | Species | Plasmid | Source | Genomic | | | Growth | of | Antibiotic | Recovery | |
| Strain | (Paenibacillus) | ID | Strain | Site | Edit Type^{b} | Tfn^{a} | at 37 C.? | integration? | Resistance? | of Edit? | Note^{c} |
| AC | polymyxa | 1 | BB | 1 | bp change | Yes | Yes | Yes | Yes | Yes | |
| AF | polymyxa | 1 | BB | 1 | bp change | Yes | Yes | Yes | Yes | No | |
| AI | polymyxa | 1 | BB | 1 | bp change | Yes | Yes | Yes | Yes | Yes | |
| AK | polymyxa | 1 | BB | 1 | bp change | Yes | Yes | No | Yes | No | (1) |
| AL | polymyxa | 1 | BB | 1 | bp change | Yes | Yes | Yes | Yes | Yes | (2) |
| AV | tritici | 1 | BB | 1 | bp change | Yes | No | | | | |
| AX | borealis | 2 | BS | 2 | truncation | Yes | No | | | | |
| AZ | borealis | 2 | BS | 2 | truncation | Yes | Yes | Yes | No | | (3) |
| BA | jilunlii | 2 | BS | 2 | truncation | No | | | | | |
| AW | sp. | 2 | BS | 2 | truncation | No | | | | | |
| AY | typhae | 2 | BS | 2 | truncation | No | | | | | |
| BR | odorifer | 3 | BT | 2 | truncation | Yes | Yes | Yes | No | | (3) |
| AA | polymyxa | 4 | BB | 1 | dup & bp change | No | | | | | |
| AB | polymyxa | 4 | BB | 1 | dup & bp change | No | | | | | |
| AC | polymyxa | 4 | BB | 1 | dup & bp change | Yes | Yes | No | | | |
| AD | polymyxa | 4 | BB | 1 | dup & bp change | Yes | Yes | Yes | Yes | | |
| AE | polymyxa | 4 | BB | 1 | dup & bp change | Yes | Yes | Yes | Yes | | |
| AF | polymyxa | 4 | BB | 1 | dup & bp change | Yes | Yes | Yes | No | | (4) |
| AG | polymyxa | 4 | BB | 1 | dup & bp change | Yes | Yes | Yes | No | | (5) |
| AH | polymyxa | 4 | BB | 1 | dup & bp change | Yes | Yes | Yes | No | | (4) |
| AI | polymyxa | 4 | BB | 1 | dup & bp change | Yes | Yes | Yes | Yes | | |
| AJ | polymyxa | 4 | BB | 1 | dup & bp change | No | | | | | |
| AL | polymyxa | 4 | BB | 1 | dup & bp change | Yes | Yes | Yes | Yes | | |
| AM | polymyxa | 4 | BB | 1 | dup & bp change | Yes | Yes | Yes | No | | (3) |
| AN | polymyxa | 4 | BB | 1 | dup & bp change | Yes | Yes | Yes | No | | (5) |
| AO | polymyxa | 4 | BB | 1 | dup & bp change | No | | | | | |
| AP | polymyxa | 4 | BB | 1 | dup & bp change | No | | | | | |
| AQ | polymyxa | 4 | BB | 1 | dup & bp change | No | | | | | |
| AA | polymyxa | 1 | BB | 1 | bp change | No | | | | | |
| AB | polymyxa | 1 | BB | 1 | bp change | No | | | | | |
| AD | polymyxa | 1 | BB | 1 | bp change | Yes | Yes | Yes | No | | (4) |
| AE | polymyxa | 1 | BB | 1 | bp change | Yes | Yes | Yes | No | | (3) |
| AG | polymyxa | 1 | BB | 1 | bp change | Yes | Yes | Yes | No | | (4) |
| AH | polymyxa | 1 | BB | 1 | bp change | Yes | Yes | Yes | No | | (3) |
| AJ | polymyxa | 1 | BB | 1 | bp change | Yes | No | | | | |
| AM | polymyxa | 1 | BB | 1 | bp change | Yes | Yes | Yes | Yes | | |
| AN | polymyxa | 1 | BB | 1 | bp change | Yes | Yes | Yes | No | | (4) |
| AO | polymyxa | 1 | BB | 1 | bp change | No | | | | | |
| AP | polymyxa | 1 | BB | 1 | bp change | No | | | | | |
| AQ | polymyxa | 1 | BB | 1 | bp change | Yes | Yes | Yes | No | | (4) |
| AC | polymyxa | 6 | BB | 1 | duplication | Yes | Yes | Yes | No | | (4) |
| AF | polymyxa | 6 | BB | 1 | duplication | Yes | Yes | Yes | No | | (4) |
| AI | polymyxa | 6 | BB | 1 | duplication | Yes | Yes | Yes | No | | (3) |
| AL | polymyxa | 6 | BB | 1 | duplication | Yes | Yes | Yes | No | | (3) |
| AA | polymyxa | 7 | AI | 2 | truncation | No | | | | | |
| AB | polymyxa | 7 | AI | 2 | truncation | No | | | | | |
| AC | polymyxa | 7 | AI | 2 | truncation | No | | | | | |
| AD | polymyxa | 7 | AI | 2 | truncation | No | | | | | |
| AE | polymyxa | 7 | AI | 2 | truncation | Yes | Yes | Yes | Yes | | |
| AF | polymyxa | 7 | AI | 2 | truncation | No | | | | | |
| AG | polymyxa | 7 | AI | 2 | truncation | Yes | Yes | Yes | Yes | | |
| AH | polymyxa | 7 | AI | 2 | truncation | No | | | | | |
| AI | polymyxa | 7 | AI | 2 | truncation | Yes | Yes | Yes | Yes | | (6) |
| AJ | polymyxa | 7 | AI | 2 | truncation | No | | | | | |
| AK | polymyxa | 7 | AI | 2 | truncation | No | | | | | |
| AL | polymyxa | 7 | AI | 2 | truncation | Yes | Yes | Yes | No | | (7) |
| AM | polymyxa | 7 | AI | 2 | truncation | Yes | Yes | Yes | No | | (7) |
| AN | polymyxa | 7 | AI | 2 | truncation | Yes | Yes | Yes | No | | (7) |
| AO | polymyxa | 7 | AI | 2 | truncation | No | | | | | |
| AP | polymyxa | 7 | AI | 2 | truncation | No | | | | | |
| AQ | polymyxa | 7 | AI | 2 | truncation | Yes | Yes | No | | | |
| BR | odorifer | 8 | AL | 3 | knockout | Yes | Yes | No | | | |
| AA | polymyxa | 8 | AL | 3 | knockout | No | | | | | |
| AB | polymyxa | 8 | AL | 3 | knockout | No | | | | | |
| AC | polymyxa | 8 | AL | 3 | knockout | Yes | Yes | Yes | Yes | | |
| AD | polymyxa | 8 | AL | 3 | knockout | Yes | Yes | No | | | |
| AE | polymyxa | 8 | AL | 3 | knockout | Yes | Yes | No | | | |
| AF | polymyxa | 8 | AL | 3 | knockout | Yes | Yes | Yes | Yes | | |
| AG | polymyxa | 8 | AL | 3 | knockout | Yes | Yes | No | | | |
| AH | polymyxa | 8 | AL | 3 | knockout | Yes | Yes | No | | | |
| AI | polymyxa | 8 | AL | 3 | knockout | Yes | Yes | No | | | |
| AJ | polymyxa | 8 | AL | 3 | knockout | Yes | Yes | No | | | |
| AK | polymyxa | 8 | AL | 3 | knockout | Yes | Yes | No | | | |
| AL | polymyxa | 8 | AL | 3 | knockout | Yes | Yes | Yes | No | | |
| AM | polymyxa | 8 | AL | 3 | knockout | Yes | Yes | Yes | Yes | | |
| AN | polymyxa | 8 | AL | 3 | knockout | Yes | Yes | No | | | |
| AO | polymyxa | 8 | AL | 3 | knockout | Yes | No | | | | |
| AP | polymyxa | 8 | AL | 3 | knockout | Yes | No | | | | |
| AQ | polymyxa | 8 | AL | 3 | knockout | Yes | No | | | | |
| BB | polymyxa | 8 | AL | 3 | knockout | No | | | | | |
| BR | odorifer | 9 | AL | 1 | bp change | Yes | Yes | No | | | |
| AA | polymyxa | 9 | AL | 1 | bp change | No | | | | | |
| AB | polymyxa | 9 | AL | 1 | bp change | No | | | | | |
| AC | polymyxa | 9 | AL | 1 | bp change | Yes | Yes | Yes | No | | |
| AD | polymyxa | 9 | AL | 1 | bp change | Yes | Yes | Yes | Yes | | |
| AE | polymyxa | 9 | AL | 1 | bp change | Yes | Yes | No | | | |
| AF | polymyxa | 9 | AL | 1 | bp change | Yes | Yes | Yes | Yes | | |
| AG | polymyxa | 9 | AL | 1 | bp change | Yes | Yes | No | | | |
| AH | polymyxa | 9 | AL | 1 | bp change | Yes | No | | | | |
| AI | polymyxa | 9 | AL | 1 | bp change | Yes | Yes | Yes | No | | |
| AJ | polymyxa | 9 | AL | 1 | bp change | Yes | Yes | Yes | Yes | | |
| AK | polymyxa | 9 | AL | 1 | bp change | Yes | Yes | No | | | |
| AL | polymyxa | 9 | AL | 1 | bp change | Yes | Yes | Yes | No | | |
| AM | polymyxa | 9 | AL | 1 | bp change | Yes | Yes | Yes | No | | |
| AN | polymyxa | 9 | AL | 1 | bp change | Yes | Yes | No | | | |
| AO | polymyxa | 9 | AL | 1 | bp change | Yes | No | | | | |
| AP | polymyxa | 9 | AL | 1 | bp change | Yes | No | | | | |
| AQ | polymyxa | 9 | AL | 1 | bp change | Yes | Yes | Yes | No | | |
| BB | polymyxa | 9 | AL | 1 | bp change | No | | | | | |
| AB | polymyxa | 10 | AL | 1 | duplication | Yes | No | | | | |
| AC | polymyxa | 10 | AL | 1 | duplication | Yes | Yes | No | | | |
| AD | polymyxa | 10 | AL | 1 | duplication | Yes | Yes | No | | | |
| AE | polymyxa | 10 | AL | 1 | duplication | Yes | Yes | Yes | Yes | | |
| AF | polymyxa | 10 | AL | 1 | duplication | Yes | Yes | Yes | Yes | | |
| AG | polymyxa | 10 | AL | 1 | duplication | Yes | Yes | No | | | |
| AH | polymyxa | 10 | AL | 1 | duplication | Yes | Yes | Yes | Yes | | |
| AJ | polymyxa | 10 | AL | 1 | duplication | Yes | No | | | | |
| AK | polymyxa | 10 | AL | 1 | duplication | Yes | Yes | No | | | |
| AM | polymyxa | 10 | AL | 1 | duplication | Yes | Yes | Yes | Yes | | |
| BK | polymyxa | 10 | AL | 1 | duplication | Yes | Yes | Yes | | | (8) |
| BO | polymyxa | 10 | AL | 1 | duplication | Yes | Yes | Yes | Yes | | |
| BR | polymyxa | 10 | AL | 1 | duplication | Yes | No | | | | |
| AB | polymyxa | 11 | AL | 1 | dup & bp change | Yes | No | | | | |
| AC | polymyxa | 11 | AL | 1 | dup & bp change | Yes | Yes | No | | | |
| AD | polymyxa | 11 | AL | 1 | dup & bp change | Yes | Yes | No | | | |
| AE | polymyxa | 11 | AL | 1 | dup & bp change | Yes | Yes | Yes | No | | (3) |
| AF | polymyxa | 11 | AL | 1 | dup & bp change | Yes | Yes | Yes | No | | (3) |
| AG | polymyxa | 11 | AL | 1 | dup & bp change | Yes | Yes | Yes | Yes | | |
| AH | polymyxa | 11 | AL | 1 | dup & bp change | Yes | Yes | No | | | |
| AJ | polymyxa | 11 | AL | 1 | dup & bp change | Yes | No | | | | |
| AK | polymyxa | 11 | AL | 1 | dup & bp change | Yes | Yes | Yes | | | (8) |
| AM | polymyxa | 11 | AL | 1 | dup & bp change | Yes | Yes | Yes | Yes | | |
| BK | polymyxa | 11 | AL | 1 | dup & bp change | Yes | Yes | Yes | | | (8) |
| BO | polymyxa | 11 | AL | 1 | dup & bp change | Yes | Yes | Yes | Yes | | |
| BR | polymyxa | 11 | AL | 1 | dup & bp change | Yes | No | | | | |

^{a}Tfn = transformation;
^{b}dup = duplication; bp = base pair
^{c}(1) lack of homology in upstream region; (2) multiple morphologies attempted; (3) all reverted to wild type; (4) PCR product for confirmation could not be obtained; (5) sequences were contaminated; (6) targeted edit; (7) PCR screen indicated all wild type; (8) PCR failed

### Example 4: Validation of Success Rates

Various mobilizable blind editing plasmids each comprising a particular edit with homology arm sequences from a particular template strain were delivered to recipient strains of the same species as the template strain. Recipient strains and conjugation competent Escherichia coli donor strains harboring the plasmids were prepared in liquid culture, mixed, and grown on solid agar medium under conditions suitable for the growth of both strains and conjugative transfer of the plasmid (mating mixtures). Mating mixtures were harvested, washed, and replated onto solid media selective for the transformed recipient strains. Recovered transconjugants were cultured under conditions selective for the chromosomal integration of the blind editing plasmids via homologous recombination. The resulting integrated strains were further cultured under conditions selective for the excision of the unwanted plasmid backbone, or the entire plasmid, from the chromosome. The edit target regions of the excised strains were sequenced to confirm proper delivery of the edit, or reversion to wild type sequence.

Seven recipient strains to which one of these edits, referred to here as Edit 4, with homology sequence derived from a particular Paenibacillus sprain here referred to as Template Strain 3, were selected for sequence homology analysis. The whole genome sequencing derived homology region sequences from these strains were identified and aligned to the Edit 4, Template Strain 3 editing cassette sequence using the Map to Reference function in Geneious Prime 2022.2.1 (Biomatters Ltd.). Homology was measured by pairwise identity for the entire approximately 2000 bp length of the homology region and for the approximately 1000 bp upstream and downstream homology regions. The differences between the percent identities of the upstream and downstream regions were calculated.

Of 165 discrete edit-to-recipient combinations tested (see Table 3), 147 combinations, or approximately 89%, yielded transconjugants (see table 3). Of these, 120, or approximately 82%, resulted in successful integration into the chromosome. Of successfully integrated strains, 89, or approximately 74%, excised under the conditions tested. Of these excised strains, 39, or approximately 44% yielded successful edits.

Sequence analysis revealed high degrees of homology for successfully blind edited strains, ranging from 100% to 97.1% pairwise identity for the entire homology region including both upstream and downstream, and ranging from 100% to 96.5% pairwise identity in the individual upstream and downstream homology arms. The maximum difference between the pairwise identities for the successfully edited strains was 1.6% (see Table 4).

Of strains that successfully received the editing plasmid via conjugation, most (approximately 82%) were able to integrate the plasmid into the chromosome via homologous recombination. Integration can occur within either the upstream or downstream homology arm and may be more likely to occur in the homology region with the highest overall homology, or the homology region with the longest sequence of complete or very high homology. For successful editing to occur, a second homologous recombination event (the excision event) must occur within the opposite homology region as the initial integration event (e.g., if the integration recombination event occurred in the upstream homology region, the excision event must occur in the downstream homology region).

Of the several steps of editing, the one with the largest rate of failure is the final edit confirmation step, where the excised strains are assayed to determine if the excision event resulted in successful delivery of the edit, or reversion to wild type. It is possible that the high rate of excision to wildtype (approximately 56%) is due to there being a higher likelihood of the excision event occurring in the same homology arm, perhaps even the same region of the homology arm, as the initial integration event. If there is a particular region of the editing cassette where integration is most likely to occur, then it is most likely that the excision event is likely to occur in that region as well.

A possible way to circumvent this bias towards excision to wild type would be to select multiple integrated colonies, each of which may have integrated by homologous recombination in a different region of the editing cassette and carry all through the steps of excision. This preserves a diversity of integration regions and increases the chances of getting initial integration in the opposite homology arm as the most likely homologous recombination event. In this case an excision event in the most likely recombination region would result in the desired edit, rather than a reversion to wild type.

Strains successfully blind edited with Edit 4 using the sequence from Template strain 3 exhibited high levels of sequence homology to the template strain, at least 97.1% over the entire editing cassette. However, the homologies of the individual homology arms could be as low as 96.5%. Since this level of homology was sufficient for a homologous recombination event to occur in that arm, theoretically a construct with this level of homology in both homology arms, yielding a total cassette homology of 96.5% identity, could yield successful blind editing. The differences between the upstream and downstream homologies of successful strains were also low in the analyzed strains. The highest difference between upstream and downstream percent identities was 1.6%. While higher differences in homology between the arms may not always prevent blind editing, it may make yielding a successful combination of integration and excision events in opposite homology arms more unlikely. In such cases, providing for a diversity of integration events and a diversity of excision events may increase the chances of a successful edit.

TABLE 2
Results

| | Strain | Paenibacillus | | Edit | | | | |
|---|---|---|---|---|---|---|---|---|
| Expt | ID | species | Edit | Template | Tfn^{a} | Integration? | Excision? | Confirmation |
| 1 | AI | polymyxa | site | BB | Yes | Yes | Yes | Edit |
| | | | inactivation | | | | | Confirmed |
| 1 | AL | polymyxa | site | BB | Yes | Yes | Yes | Edit |
| | | | inactivation | | | | | Confirmed |
| 1 | AF | polymyxa | site | BB | Yes | Yes | Yes | WT |
| | | | inactivation | | | | | |
| 1 | AK | polymyxa | site | BB | Yes | Yes | Yes | WT |
| | | | inactivation | | | | | |
| 1 | AV | polymyxa | site | BB | Yes | No | | |
| | | | inactivation | | | | | |
| 1 | AC | polymyxa | site | BB | Yes | Yes | Yes | Edit |
| | | | inactivation | | | | | Confirmed |
| 2 | AB | polymyxa | site dup & | BB | No | | | |
| | | | inactivation | | | | | |
| 2 | AC | polymyxa | site dup & | BB | Yes | Yes | No | |
| | | | inactivation | | | | | |
| 2 | AD | polymyxa | site dup & | BB | Yes | Yes | Yes | Edit |
| | | | inactivation | | | | | Confirmed |
| 2 | AE | polymyxa | site dup & | BB | Yes | Yes | Yes | Edit |
| | | | inactivation | | | | | Confirmed |
| 2 | AF | polymyxa | site dup & | BB | Yes | Yes | Yes | WT |
| | | | inactivation | | | | | |
| 2 | AG | polymyxa | site dup & | BB | Yes | Yes | Yes | WT |
| | | | inactivation | | | | | |
| 2 | AH | polymyxa | site dup & | BB | Yes | Yes | Yes | WT |
| | | | inactivation | | | | | |
| 2 | AI | polymyxa | site dup & | BB | Yes | Yes | Yes | Edit |
| | | | inactivation | | | | | Confirmed |
| 2 | AJ | polymyxa | site dup & | BB | No | | | |
| | | | inactivation | | | | | |
| 2 | AX | borealis | truncation | BS | Yes | No | | |
| 2 | AK | polymyxa | site dup & | BB | Yes | Yes | Yes | Edit |
| | | | inactivation | | | | | Confirmed |
| 2 | AL | polymyxa | site dup & | BB | Yes | Yes | Yes | Edit |
| | | | inactivation | | | | | Confirmed |
| 2 | AM | polymyxa | dup | AL | Yes | Yes | Yes | Edit |
| | | | | | | | | Confirmed |
| 2 | AO | polymyxa | site dup & | BB | No | | | |
| | | | inactivation | | | | | |
| 2 | AP | polymyxa | site dup & | BB | No | | | |
| | | | inactivation | | | | | |
| 2 | AQ | polymyxa | site dup & | BB | Yes | Yes | Yes | WT |
| | | | inactivation | | | | | |
| 2 | AA | polymyxa | site | BB | No | | | |
| | | | inactivation | | | | | |
| 2 | AO | polymyxa | site | BB | No | | | |
| | | | inactivation | | | | | |
| 2 | AP | polymyxa | site | BB | No | | | |
| | | | inactivation | | | | | |
| 2 | AQ | polymyxa | site | BB | Yes | No | | |
| | | | inactivation | | | | | |
| 2 | AB | polymyxa | site | BB | No | | | |
| | | | inactivation | | | | | |
| 2 | AD | polymyxa | site | BB | Yes | Yes | Yes | WT |
| | | | inactivation | | | | | |
| 2 | AE | polymyxa | site | BB | Yes | Yes | Yes | WT |
| | | | inactivation | | | | | |
| 2 | AG | polymyxa | site | BB | Yes | Yes | Yes | WT |
| | | | inactivation | | | | | |
| 2 | AH | polymyxa | site | BB | Yes | Yes | Yes | WT |
| | | | inactivation | | | | | |
| 2 | AJ | polymyxa | site | BB | Yes | Yes | Yes | WT |
| | | | inactivation | | | | | |
| 2 | AM | polymyxa | site dup & | AL | Yes | Yes | Yes | Edit |
| | | | inactivation | | | | | Confirmed |
| 2 | BU | borealis | truncation | BS | Yes | Yes | Yes | WT |
| 2 | AN | polymyxa | truncation | AL | Yes | Yes | Yes | WT |
| 2 | AC | polymyxa | dup | BB | Yes | Yes | Yes | WT |
| 2 | AF | polymyxa | dup | BB | Yes | Yes | Yes | WT |
| 2 | AI | polymyxa | dup | BB | Yes | Yes | Yes | WT |
| 2 | AK | polymyxa | dup | BB | Yes | Yes | Yes | WT |
| 2 | AL | polymyxa | dup | BB | Yes | Yes | Yes | WT |
| 2 | BR | odorifer | site dup & | AL | Yes | No | | |
| | | | inactivation | | | | | |
| 2 | AA | polymyxa | site dup & | BB | No | | | |
| | | | inactivation | | | | | |
| 3 | AB | polymyxa | knockout | AL | No | | | |
| 3 | AF | polymyxa | site | AL | Yes | Yes | Yes | Edit |
| | | | inactivation | | | | | Confirmed |
| 3 | AG | polymyxa | knockout | AL | Yes | Yes | No | |
| 3 | AG | polymyxa | site | AL | Yes | Yes | No | |
| | | | inactivation | | | | | |
| 3 | AH | polymyxa | knockout | AL | Yes | Yes | No | |
| 3 | AH | polymyxa | site | AL | Yes | No | | |
| | | | inactivation | | | | | |
| 3 | AI | polymyxa | knockout | AL | Yes | Yes | No | |
| 3 | AI | polymyxa | site | AL | Yes | Yes | Yes | WT |
| | | | inactivation | | | | | |
| 3 | AJ | polymyxa | knockout | AL | Yes | Yes | No | |
| 3 | AJ | polymyxa | site | AL | Yes | Yes | Yes | Edit |
| | | | inactivation | | | | | Confirmed |
| 3 | AK | polymyxa | knockout | AL | Yes | Yes | No | |
| 3 | AB | polymyxa | site | AL | No | | | |
| | | | inactivation | | | | | |
| 3 | AK | polymyxa | site | AL | Yes | Yes | No | |
| | | | inactivation | | | | | |
| 3 | AL | polymyxa | truncation | BB | Yes | Yes | Yes | Edit |
| | | | | | | | | Confirmed |
| 3 | BO | polymyxa | dup | AL | Yes | Yes | Yes | Edit |
| | | | | | | | | Confirmed |
| 3 | AN | polymyxa | truncation | AL | Yes | Yes | Yes | WT |
| 3 | BV | polymyxa | dup | AL | Yes | Yes | Yes | WT |
| 3 | BR | odorifer | truncation | AL | Yes | No | | |
| 3 | AC | polymyxa | knockout | AL | Yes | Yes | Yes | Edit |
| | | | | | | | | Confirmed |
| 3 | AC | polymyxa | site | AL | Yes | Yes | Yes | WT |
| | | | inactivation | | | | | |
| 3 | AD | polymyxa | knockout | AL | Yes | Yes | No | |
| 3 | AD | polymyxa | site | AL | Yes | Yes | Yes | Edit |
| | | | inactivation | | | | | Confirmed |
| 3 | AE | polymyxa | knockout | AL | Yes | Yes | No | |
| 3 | AE | polymyxa | site | AL | Yes | Yes | No | |
| | | | inactivation | | | | | |
| 3 | AF | polymyxa | knockout | AL | Yes | Yes | Yes | Edit |
| | | | | | | | | Confirmed |
| 4 | AA | polymyxa | knockout | AL | No | | | |
| 4 | AQ | polymyxa | site | AL | Yes | Yes | Yes | WT |
| | | | inactivation | | | | | |
| 4 | AA | polymyxa | site | AL | No | | | |
| | | | inactivation | | | | | |
| 4 | BB | polymyxa | knockout | AL | No | | | |
| 4 | BB | polymyxa | site | AL | No | | | |
| | | | inactivation | | | | | |
| 4 | AO | polymyxa | knockout | AL | Yes | No | | |
| 4 | AO | polymyxa | site | AL | Yes | No | | |
| | | | inactivation | | | | | |
| 4 | AP | polymyxa | knockout | AL | Yes | No | | |
| 4 | AP | polymyxa | site | AL | Yes | No | | |
| | | | inactivation | | | | | |
| 4 | AQ | polymyxa | knockout | AL | Yes | No | | |
| 5 | AB | polymyxa | dup | AL | Yes | No | | |
| 5 | BK | polymyxa | dup | AL | Yes | Yes | Yes | WT |
| 5 | AB | polymyxa | site dup & | AL | Yes | No | | |
| | | | inactivation | | | | | |
| 5 | AD | polymyxa | site dup & | AL | Yes | Yes | No | |
| | | | inactivation | | | | | |
| 5 | AE | polymyxa | site dup & | AL | Yes | Yes | Yes | WT |
| | | | inactivation | | | | | |
| 5 | AG | polymyxa | site dup & | AL | Yes | Yes | Yes | Edit |
| | | | inactivation | | | | | Confirmed |
| 5 | AH | polymyxa | site dup & | AL | Yes | Yes | No | |
| | | | inactivation | | | | | |
| 5 | AJ | polymyxa | site dup & | AL | Yes | No | | |
| | | | inactivation | | | | | |
| 5 | AK | polymyxa | site dup & | AL | Yes | Yes | Yes | WT |
| | | | inactivation | | | | | |
| 5 | BO | polymyxa | site | BB | Yes | Yes | Yes | Edit |
| | | | inactivation | | | | | Confirmed |
| 5 | BK | polymyxa | site dup & | AL | Yes | Yes | Yes | WT |
| | | | inactivation | | | | | |
| 5 | AD | polymyxa | dup | AL | Yes | Yes | No | |
| 5 | AE | polymyxa | dup | AL | Yes | Yes | Yes | Edit |
| | | | | | | | | Confirmed |
| 5 | AF | polymyxa | dup | AL | Yes | Yes | Yes | Edit |
| | | | | | | | | Confirmed |
| 5 | AG | polymyxa | dup | AL | Yes | Yes | No | |
| 5 | AH | polymyxa | dup | AL | Yes | Yes | Yes | Edit |
| | | | | | | | | Confirmed |
| 5 | AJ | polymyxa | dup | AL | Yes | No | | |
| 5 | AK | polymyxa | dup | AL | Yes | Yes | No | |
| 6 | BW | polymyxa | dup | AL | No | | | |
| 6 | BX | graminis | site dup & | AL | Yes | Yes | Yes | WT |
| | | | inactivation | | | | | |
| 6 | BY | polymyxa | site dup & | AL | Yes | Yes | Yes | WT |
| | | | inactivation | | | | | |
| 6 | BZ | polymyxa | site dup & | AL | Yes | Yes | Yes | WT |
| | | | inactivation | | | | | |
| 6 | CA | polymyxa | site dup & | AL | No | | | |
| | | | inactivation | | | | | |
| 6 | BR | odorifer | dup | AL | Yes | No | | |
| 6 | CB | polymyxa | dup | AL | Yes | No | | |
| 6 | BX | graminis | dup | AL | No | | | |
| 6 | BY | polymyxa | dup | AL | Yes | Yes | Yes | WT |
| 6 | BZ | polymyxa | dup | AL | Yes | Yes | Yes | WT |
| 6 | CA | polymyxa | dup | AL | Yes | Yes | Yes | Edit |
| | | | | | | | | Confirmed |
| 6 | BW | polymyxa | site dup & | AL | Yes | Yes | No | |
| | | | inactivation | | | | | |
| 6 | CB | polymyxa | site dup & | AL | Yes | No | | |
| | | | inactivation | | | | | |
| 7 | AM | polymyxa | site | AL | Yes | Yes | Yes | WT |
| | | | inactivation | | | | | |
| 7 | AN | polymyxa | site | AL | Yes | Yes | No | |
| | | | inactivation | | | | | |
| 8 | BV | polymyxa | site dup & | AL | Yes | Yes | No | |
| | | | inactivation | | | | | |
| 8 | BO | polymyxa | site dup & | AL | Yes | Yes | Yes | Edit |
| | | | inactivation | | | | | Confirmed |
| 8 | AB | polymyxa | truncation | AL | Yes | No | | |
| 8 | AN | polymyxa | site dup & | BB | Yes | Yes | Yes | WT |
| | | | inactivation | | | | | |
| 8 | AE | polymyxa | truncation | AL | Yes | Yes | Yes | Edit |
| | | | | | | | | Confirmed |
| 8 | AI | polymyxa | truncation | AL | Yes | Yes | Yes | Edit |
| | | | | | | | | Confirmed |
| 8 | AK | polymyxa | truncation | AL | Yes | No | | |
| 9 | BB | polymyxa | truncation | AL | Yes | No | | |
| 9 | AF | polymyxa | truncation | AL | Yes | Yes | No | |
| 9 | AG | polymyxa | truncation | AL | Yes | Yes | Yes | WT |
| 9 | AH | polymyxa | truncation | AL | Yes | Yes | Yes | WT |
| 9 | BK | polymyxa | truncation | AL | Yes | Yes | Yes | WT |
| 9 | AJ | polymyxa | truncation | AL | Yes | Yes | No | |
| 9 | AN | polymyxa | site | BB | Yes | Yes | Yes | WT |
| | | | inactivation | | | | | |
| 9 | AN | polymyxa | knockout | AL | Yes | Yes | No | |
| 9 | BR | odorifer | truncation | BT | Yes | Yes | Yes | WT |
| 9 | AC | polymyxa | truncation | AL | Yes | Yes | No | |
| 9 | AD | polymyxa | truncation | AL | Yes | Yes | Yes | WT |
| 10 | AM | polymyxa | site dup & | BB | Yes | Yes | Yes | WT |
| | | | inactivation | | | | | |
| 10 | AM | polymyxa | site | BB | Yes | Yes | Yes | Edit |
| | | | inactivation | | | | | Confirmed |
| 10 | BO | polymyxa | site | AL | Yes | Yes | Yes | Edit |
| | | | inactivation | | | | | Confirmed |
| 10 | BO | polymyxa | truncation | AL | Yes | Yes | Yes | WT |
| 11 | CC | polymyxa | site | AL | Yes | Yes | No | |
| | | | inactivation | | | | | |
| 11 | CC | polymyxa | dup | AL | Yes | Yes | Yes | Edit |
| | | | | | | | | Confirmed |
| 11 | CC | polymyxa | site dup & | AL | Yes | Yes | Yes | Edit |
| | | | inactivation | | | | | Confirmed |
| 12 | CD | polymyxa | site | AL | Yes | Yes | No | |
| | | | inactivation | | | | | |
| 12 | CD | polymyxa | dup | AL | Yes | Yes | No | |
| 12 | CD | polymyxa | site dup & | AL | Yes | Yes | Yes | WT |
| | | | inactivation | | | | | |
| 13 | AF | polymyxa | site dup & | AL | Yes | Yes | Yes | Edit |
| | | | inactivation | | | | | Confirmed |
| 13 | AM | polymyxa | knockout | AL | Yes | Yes | Yes | Edit |
| | | | | | | | | Confirmed |
| 13 | BO | polymyxa | truncation | AL | Yes | Yes | Yes | WT |
| 14 | AC | polymyxa | dup | AL | Yes | Yes | Yes | Edit |
| | | | | | | | | Confirmed |
| 14 | AC | polymyxa | site dup & | AL | Yes | Yes | Yes | Edit |
| | | | inactivation | | | | | Confirmed |
| 15 | CE | polymyxa | site | BB | Yes | Yes | No | |
| | | | inactivation | | | | | |
| 15 | CF | polymyxa | site | AL | Yes | Yes | Yes | WT |
| | | | inactivation | | | | | |
| 15 | CF | polymyxa | truncation | AI | Yes | Yes | No | |
| 15 | CF | polymyxa | truncation | AL | Yes | Yes | Yes | WT |
| 15 | CG | polymyxa | site | BB | Yes | Yes | Yes | WT |
| | | | inactivation | | | | | |
| 15 | CG | polymyxa | site | AL | Yes | Yes | No | |
| | | | inactivation | | | | | |
| 15 | CG | polymyxa | truncation | AI | Yes | Yes | No | |
| 15 | CG | polymyxa | truncation | AL | Yes | Yes | Yes | Edit |
| | | | | | | | | Confirmed |
| 15 | CH | polymyxa | site | BB | Yes | No | | |
| | | | inactivation | | | | | |
| 15 | CH | polymyxa | site | AL | Yes | Yes | Yes | Edit |
| | | | inactivation | | | | | Confirmed |
| 15 | CH | polymyxa | truncation | AI | Yes | No | | |
| 15 | CE | polymyxa | site | AL | Yes | Yes | Yes | Edit |
| | | | inactivation | | | | | Confirmed |
| 15 | CH | polymyxa | truncation | AL | Yes | No | | |
| 15 | CE | polymyxa | truncation | AI | Yes | Yes | No | |
| 15 | CE | polymyxa | truncation | AL | Yes | Yes | Yes | Edit |
| | | | | | | | | Confirmed |
| 15 | CI | polymyxa | site | BB | Yes | Yes | Yes | Edit |
| | | | inactivation | | | | | Confirmed |
| 15 | CI | polymyxa | site | AL | Yes | Yes | Yes | WT |
| | | | inactivation | | | | | |
| 15 | CI | polymyxa | truncation | AI | Yes | No | | |
| 15 | CI | polymyxa | truncation | AL | Yes | No | | |
| 15 | CF | polymyxa | site | BB | Yes | Yes | Yes | WT |
| | | | inactivation | | | | | |
| 16 | CJ | polymyxa | truncation | BU | Yes | No | | |
| 16 | CK | graminis | truncation | BU | Yes | Yes | Yes | WT |

TABLE 3
Success Rates

| | Transformation | Integration | Excision | Edit Confirmed |
|---|---|---|---|---|
| Success | 147 | 120 | 89 | 40 |
| Failure | 18 | 27 | 31 | 50 |
| Total | 165 | 147 | 120 | 90 |

TABLE 4
Example Sequence Homologies as Measured by Percent Identities

| | | | | Upstream and |
|---|---|---|---|---|
| Recipient | Global | Upstream | Downstream | Downstream |
| Strain | Homology | Homology | Homology | Difference |
| AI | 99.3 | 99.8 | 98.8 | 1 |
| AF | 97.1 | 96.5 | 98.1 | 1.6 |
| AC | 98.1 | 98.1 | 98.4 | 0.3 |
| AE | 99.3 | 99.8 | 98.8 | 1 |
| AH | 97.1 | 96.5 | 98.1 | 1.6 |
| BO | 100 | 100 | 100 | 0 |
| CC | 99.3 | 99.8 | 98.8 | 1 |

1. A method of introducing an edit into a target site of a recipient polynucleotide, the method comprising:
(a) determining the composition of a source polynucleotide;
(b) designing an introductory polynucleotide, wherein the introductory polynucleotide comprises a region that shares sufficient homology with at least 100 nucleotides of the source polynucleotide of (a);
wherein at least one of the following conditions is true:
(i) the sequence of recipient polynucleotide is undetermined, and/or
(ii) the sequence of recipient polynucleotide is not 100% identical to the sequence of source polynucleotide:
(c) providing the introductory polynucleotide to the recipient polynucleotide;
(d) incubating the introductory polynucleotide and the recipient polynucleotide under conditions suitable for recombining; and
(e) assessing the recipient polynucleotide for at least one edit at the target site.

2. The method of claim 1, further comprising:
(f) sequencing the recipient polynucleotide.

3. The method of claim 1, wherein the source polynucleotide and recipient polynucleotide are alleles, homologs, orthologs, or paralogs.

4. The method of claim 1, wherein the recipient polynucleotide does not comprise a coding region.

5. The method of claim 1, wherein the source polynucleotide and recipient polynucleotide are comprised within the same organism.

6. The method of claim 1, wherein the composition of the source polynucleotide is determined by generating a consensus sequence from a plurality of sequences.

7. The method of claim 1, wherein the introductory polynucleotide further comprises a polynucleotide of interest, wherein the polynucleotide of interest is flanked by the sequences sharing sufficient homology with at least 100 nucleotides up- and down-stream, respectively, of the source polynucleotide.

8. The method of claim 6, wherein the polynucleotide of interest comprises a heterologous polynucleotide that is inserted into a target site of the recipient polynucleotide, and/or a polynucleotide modification template.

9. (canceled)

10. The method of claim 1, wherein the edit is selected from the group consisting of: insertion of at least one nucleotide, deletion of at least one nucleotide, replacement of at least one nucleotide, molecular alteration of at least one nucleotide, and any combination of the preceding.

11. The method of claim 1, wherein the edit results in the increased expression of a gene, the decreased expression of a gene, the inactivation of a gene, the knockout of a gene, or the expression of a new gene.

12. The method of claim 1, wherein sufficient homology is at least 80% identity over at least 100 nucleotides and/or successful homologous recombination between the recipient polynucleotide and the introduced polynucleotide.

13. (canceled)

14. The method of claim 1, wherein the source polynucleotide is selected from the group consisting of: from the same species of cell as the recipient polynucleotide, from a different Genus than the cell of the recipient polynucleotide, from a different species than the cell of the recipient polynucleotide, and from a synthetic source.

15. (canceled)

16. (canceled)

17. The method of claim 1, wherein the recipient polynucleotide is in the genome of a cell.

18. The method of claim 14, further comprising:
(f) incubating the cell under conditions that facilitate growth and reproduction.

19. The method of claim 1, comprising a plurality of recipient polynucleotides.

20. The method of claim 18, wherein at least two of the plurality of recipient polynucleotides are in the genomes of different cells.

21. The method of claim 19, wherein the different cells comprise bacterial cells.

22. The method of claim 20, wherein at least one bacterial cell is of a different species or a different strain of the same species.

23. (canceled)

24. The method of claim 18, wherein the plurality of recipient polynucleotides are in vitro or ex vivo.

25. The method of claim 1, comprising a plurality of source polynucleotides.

26. A synthetic composition, comprising:
(a) an introductory polynucleotide, comprising a region that shares sufficient homology with at least 100 nucleotides of a source polynucleotide; and
(b) a plurality of recipient polynucleotides;
wherein the plurality of recipient polynucleotides are not all identical;
wherein the source polynucleotide is identified or obtained from a source organism that is of the same species as the organism from which at least one of the recipient polynucleotide(s) is (are) identified or obtained;
wherein at least one of the following conditions is true:
(i) the sequence of at least one recipient polynucleotide is undetermined, and/or
(ii) the sequence of at least one recipient polynucleotide is not 100% identical to the sequence of source polynucleotide.

27. The synthetic composition of claim 25, wherein the plurality of recipient polynucleotides are comprised within a plurality of cells.

28. The synthetic composition of claim 25, wherein the source polynucleotide is comprised within a cell.

29. The synthetic composition of claim 25, comprising a plurality of source polynucleotides.

30. A synthetic composition, comprising:
(c) a plurality of introductory polynucleotides, each comprising a region that shares sufficient homology with at least 100 nucleotides of a source polynucleotide; and
(d) one or more recipient polynucleotide(s);
wherein the plurality of source polynucleotides are not all identical;
wherein the source polynucleotide is:
(i) identified or obtained from a source organism that is of the same species as the organism from which at least one of the recipient polynucleotide(s) is (are) identified or obtained; or
(ii) obtained from a consensus sequence of a plurality of polynucleotides:
wherein at least one of the following conditions is true:
(i) the sequence of at least one recipient polynucleotide is undetermined, and/or
(ii) the sequence of at least one recipient polynucleotide is not 100% identical to the sequence of at least one source polynucleotide.

31. A kit comprising:
(a) a plurality of introductory polynucleotides, each comprising a region that shares sufficient homology with at least 200 nucleotides of a source polynucleotide, wherein the plurality of introductory polynucleotides are not all identical; and
(b) a composition that promotes stability of the introductory polynucleotides.
